(19) United States
(12) Patent Application Publication
(10) Pub. No.: US 2026/0071261 A1
(43) Pub. Date: Mar. 12, 2026

(54) METHOD OF ASSESSING PROTEIN PRODUCTION IN CHO CELLS

(71) Applicant: Evonik Operations GmbH, Essen (DE)

(72) Inventors: Suki ROY, Sydney, New South Wales (AU); Sanjanaa NAGARAJAN, Singapore (SG); Florian BÖHL, Neckargemünd (DE); Lingzhi HUANG, Singapore (SG); Sarah CHAN, Bellevue, WA (US); Kit Yeng WONG, Songapore (SG); Rose WHELAN, Ambrosden Oxfordshire (GB); Daniel FRANKE, Essen (DE)

(73) Assignee: Evonik Operations GmbH, Essen (DE)

(21) Appl. No.: 19/107,425

(22) PCT Filed: Aug. 23, 2023
(86) PCT No.: PCT/EP2023/073125

§ 371 Date: Feb. 27, 2025

(30) Foreign Application Priority Data
Sep. 1, 2022 (EP) 22193449.0

(51) Int. Cl. C12Q 1/6827; C12Q 1/6809; C12Q 1/6881

(52) U.S. Cl. CPC C12Q 1/6827; C12Q 1/6809; C12Q 1/6881; C12Q 2600/158

(57) ABSTRACT

The present invention is related to a method of determining suitability of at least one Chinese Hamster Ovary (CHO) test cell line for optimal heterologous protein production, the method comprising:
(a) determining a test methylation profile from genomic material obtained from the CHO test cell line; and
(b) comparing the test methylation profile obtained from (a) with a reference methylation profile, wherein the reference methylation profile comprises the methylation status of more than one CpG site from at least one CHO reference cell line that displays at least one phenotype of interest for optimal heterologous protein production,
   wherein a significant similarity in the test methylation profile of (a) compared to the reference methylation profile, is indicative of the CHO test cell line being suitable for optimal heterologous protein production and wherein the test methylation profile and reference methylation profile are from CpG sites from the CHO cell genome and are determined using DNA methylation-bead-based array.

### CROSS REFERENCE TO RELATED APPLICATIONS

This application is a National Stage of International Application No. PCT/EP2023/073125 filed Aug. 23, 2023, claiming priority based on European 22193449.0 filed Sep. 1, 2022.

### FIELD OF THE INVENTION

The present invention relates to a method based on epigenetics for quantitatively and qualitatively assessing target protein production in CHO cells and cell stability prior, during or after the actual production of the protein. In particular, the measure of differential methylation of promotors and/or CpG sites of CHO cells may provide an insight into the quantitative and qualitative production of the target protein by the CHO cells.

### BACKGROUND OF THE INVENTION

Chinese Hamster Ovary (CHO) cells are known to be the workhorses for the industrial production of recombinant therapeutic proteins since 1987 and are hence widely used for biologics production. About 70% of all recombinant biopharmaceutical proteins and all monoclonal antibodies approved since 2016 are being manufactured in CHO cells. Several advantages of utilizing CHO for biologics production include tolerance to genetic manipulations, ease of adaptation to manufacturing process scales, rapid growth rates, and ability to perform human-compatible post-translational modifications. However, the biologics production system in CHO faces a bottleneck due to the loss of protein productivity over time.

Initial protein expression from the cell line is high, however the production reduces during prolonged culture. This results in decreased process yield, impacts timelines and increases costs. Changes in cell culture environment can result in an alteration of cell behaviour and protein productivity of the producer cell line. A few reasons for loss of productivity in CHO cells include accumulation of large numbers of genomic variations over prolonged culture, loss of transgene and epigenetic regulation of transgene insertion sites and the like. In particular, the integration sites of the viral promoter are susceptible to transcriptional regulation via epigenetic regulation such as histone modifications and DNA methylation. In particular, the DNA methylation status of the viral promoter is an important factor in protein production or expression stability in producer CHO cells. An increase in DNA methylation in the promoter results in transgene silencing at transcription levels. The protein production variability in CHO cells has been associated with DNA methylation mediated regulation of Cytomegalovirus Major Immediate-Early and enhancer (CMV) promoter and simian vacuolating virus 40 (SV40) promoters which are the most frequently used promoters for the production of recombinant proteins in CHO cells.

The current methods of determining the suitability of a CHO clone for target protein production are not only time-consuming but also not very accurate for selection of clones or cells for optimal protein production.

Further, genetically identical CHO clones can still result in heterogenous phenotypes, creating instability, inefficiency and financial loss during heterologous protein production at an industrial scale. Methods to compare and select CHO clones that use only phenotypic analyses are not able to guarantee consistency over time. Genotype comparisons of CHO clones cannot define the how genes are expressed differentially to adapt to environmental conditions. As shown by Wippermann A, et al., Appl Microbiol Biotechnol. 2014 January; 98 (2): 579-89, supplementation of butyrate which is known to enhance cell specific productivities in CHO cells also led to alterations of epigenetic silencing events.

Accordingly, there is a need in the art for a tool that is efficient and affordable to globally evaluate and regulate CHO metabolism and protein production. There is also a need in the art for methods of selection and maintenance of identical CHO populations in order to improve speed, quality, efficiency and consistency of production.

### BRIEF DESCRIPTION OF FIGURES

FIG. 1 is a plot showing the results of Principle Component Analysis (PCA) of 122 differentially methylated regions (DMRs) identified.

FIG. 2 is a plot showing the results of Principle Component Analysis (PCA) of 289 differentially methylated regions (DMRs) identified.

FIG. 3 is a graph showing the live cell count of control CHO Humira431 cells and hyperosmolality-treated CHO Humira431 cells. Sodium Chloride was added to hyperosmolality-treated CHO Humira431 cells on day 3. From day 3 onwards, a stagnation of live cell count can be seen in hyperosmolality-treated CHO Humira431 cells, as opposed to control CHO Humira431, which continued to increase until day 10, when the live cell count begins to plateau.

FIG. 4 is a graph showing the heterologous protein productivity of hyperosmolality-treated CHO Humira431 cells and control CHO Humira431 cells on day 7 of the fed-batch culture. Hyperosmolality-treated CHO Humira431 cells were found to produce between 86.5 pg/cell to 90.4 pg/cell heterologous protein, as opposed to control CHO Humira431 cells, which produced between 40.4 pg/cell to 41.3 pg/cell heterologous protein. Addition of Sodium Chloride to hyperosmolality-treated CHO Humira431 cells was therefore found to result in increased heterologous protein productivity on day 7 of the fed-batch culture.

FIG. 5 is a graph showing classification of 6 clones based on the on heterologous protein productivity on day 9, 11 and 14 of the fed batch culture. Based on productivity, clone 2C9, 3D11, 2H2 are classified as low producers, clone 10A8 is classified as intermediate producer and clone 7H9 and 8F8 are classified as high producers.

FIG. 6 is PCA plot showing the clustering of groups based on protein productivity

### DESCRIPTION OF THE INVENTION

The present invention solves the problems above by providing a means of not only identifying genetically identical CHO clones or cell lines but confirming that these clones and/or cell lines are phenotypically homogenous thus ensuring stability, efficiency and reduction of financial loss during heterologous protein production particularly at an industrial scale. In particular, the methods according to any aspect of the present invention use methylation patterns and conservation of these methylation patterns in CHO clones and/or cell lines for selection and maintenance of identical CHO populations in order to improve speed, quality, efficiency and consistency of heterologous protein production. Since genotype comparisons of CHO clones cannot define the how genes are expressed differentially to adapt to environmental conditions, and phenotypic analyses alone are not able to guarantee consistency over time, epigenetic methods, specifically DNA methylation therefore provides a state-of-the-art technology to select not only genetically identical, but epigenetically and therefore phenotypically identical CHO clones for improved heterologous protein production. The method according to any aspect of the present invention allow the use of DNA methylation as a tool to improve protein production quantitatively and qualitatively from CHO cells. Altering DNA methylation pattern on viral promoter driving transgene expression will transcriptionally increase protein expression in CHO cells.

According to one aspect of the present invention, there is provided a method of determining suitability of at least one Chinese Hamster Ovary (CHO) test cell line for optimal heterologous protein production, the method comprising:
(a) determining a test methylation profile from genomic material obtained from the CHO test cell line; and
(b) comparing the test methylation profile obtained from (a) with a reference methylation profile, wherein the reference methylation profile comprises the methylation status of more than one CpG site from at least one CHO reference cell line that displays at least one phenotype of interest for optimal heterologous protein production,
   wherein a significant similarity in the test methylation profile of (a) compared to the reference methylation profile, is indicative of the CHO test cell line being suitable for optimal heterologous protein production and wherein the test methylation profile and reference methylation profile are from CpG sites from the CHO cell genome and are determined using DNA methylation-bead-based array.

Epigenetics technologies thus provides a solution for the quantitative and qualitative analysis of protein production. In particular, the reference methylation profile may comprise environmental specific CpG sites or dynamic CpG sites i.e., sites which seem to have a crucial role in several environmental conditions; CpG sites in the viral promoters (CMV and SV40 promoters) and/or CpG sites from regulatory regions of candidate genes from pathways which are significant in certain important biological processes for the CHO cell (e.g. metabolic linked genes, protein production linked genes, cell growth/division linked genes, and methylation linked genes). More in particular, the test and reference methylation profiles are from CpG sites from wherein the test methylation profile and reference methylation profile are from CpG sites from the CHO cell genome.

The term ‘CHO cell genome’ herein refers to the genomic DNA of the CHO cell that excludes the DNA of a virus, particularly CMV and SV40, that are used to introduce foreign DNA to the cell. In particular, the CHO cell genome may denote the cell with a genome make-up that is in a form as seen naturally in the wild. The term may also include genes which have been added to the CHO genome by genetic modification (i.e. with regard to improved production of protein etc.) but not necessarily or not genes and promoters of viruses that have been used to introduce the genes into the CHO genome. The term “CHO cell genome” therefore may exclude virus genes and promoters and/or may include endogenous or homologous genes of the CHO cell and/or genetically modified endogenous or homologous genes of the CHO cell and/or intergenic genes, DNA found between the genes of the CHO cell.

The method according to any aspect of the present invention may be used to quantity the methylation level at any one of these CpG sites for the CHO cells, particularly the test CHO cells. This information can then be used to assess, evaluate and enhance the CHO cells phenotypically in various cell culture conditions. More in particular, machine learning models may be used to analyse the quantitative and qualitative methylation data generated. Even more in particular, the methods according to any aspect of the present invention may be used in a predictive and precise way for designing optimal cell culture conditions, especially in terms of selection of the suitable CHO cell line, compared to the current methods of trial and error that are used. This thus allows the online and direct control of manufacturing processes, increasing the robustness and thus overall, the quality of molecules produced by the CHO cells.

The CHO cell line refers to immortal Chinese Hamster Ovary cell line (CHO) derived from Cricetulus griseus. In particular, the CHO cell line may be selected from the group consisting of CHO-K1 (ATCC), CHO-DG44 (Thermo Fisher Scientific), CHO-DXB11 (ATCC), ExpiCHO-S™ cells (Thermo Fisher Scientific), FreeStyle™ CHO-S™ cells (Thermo Fisher Scientific), CHO 1-15 [subscript 500] (ATCC) and Agarabi CHO (ATCC).

The term ‘suitability’ as used herein, refers to a CHO cell line that is fit for optimal heterologous protein production. In one example, a CHO cell line may be considered suitable for optimal heterologous protein production before a transgene is introduced into the cell. In this case, the CHO cell line may have phenotypic parameters or characteristics that enable the cell line to grow well and allow for easy uptake of the transgene of interest and following the uptake of the transgene, allow for optimal heterologous protein production, where the protein is a product of the transgene of interest. These characteristics or phenotypic parameters include at least optimal glucose consumption, growth rate, lactic acid production, ammonia accumulation and the like. When a CHO cell line is confirmed of displaying at least one of these phenotypic parameters, the CHO cell line may be considered suitable for optimal heterologous protein production when the transgene of interest is introduced into the cell.

In another example, a CHO cell line may be considered suitable for optimal heterologous protein production after the transgene has been introduced into the cell. In this case, a CHO cell line is genetically modified using methods known in the art to introduce a transgene into the cell and the genetically modified cell is capable of optimal heterologous protein production where the protein is a product of translation of the transgene. The CHO cell line in this example, may have a least one phenotype of interest that enables the genetically modified cell line to have good viability and optimal target protein production. These phenotypes of interest may include cell viability (survivability), protein productivity (in terms of protein quantity and quality), phenotypic homogeneity, cell exhaustion, and the like. Accordingly, the method according to any aspect of the present invention may be used on a CHO cell line that has been genetically modified (i.e. with transgene introduced into the cell line) or on a CHO cell line that has not yet been genetically modified. In both cases, the CHO cell lines for use in heterologous protein production.

As used herein, the term ‘transgene’ refers to a gene that is taken from the genome of one organism and inserted into the genome of another organism by artificial techniques used in genetic modification. For example, a human gene is artificially introduced into the genome of CHO cells for the production of at least one protein of interest, particularly therapeutic proteins.

As used herein, the term ‘therapeutic protein’ refers to genetically engineered versions of naturally occurring human proteins. Examples of therapeutic proteins include antibody-based drugs, anticoagulants, blood factors, bone morphogenetic proteins, engineered protein scaffolds, enzymes, growth factors, hormones, interferons, interleukins and the like.

As used herein, the term ‘cell survivability’ refers to the capability of a cell to be viable and perform cell proliferation. Cell viability is a measure of the proportion of live cells within a population. Cell proliferation refers to an increase in cell number due to cell division. The assays that are commonly used to test cell survivability include BrdU Cell Proliferation Assay, MTT Cell Proliferation Assays, trypan blue cell counting, and ATP Cell Viability Assays.

As used herein, the term ‘cell exhaustion’ refers to the state of the cell where it loses its capability to perform metabolic activity including heterologous protein production. Cell exhaustion can be determined by Metabolite Detection Assays.

As used herein, the term ‘phenotypic homogeneity’ refers to a state when all the cells in a population exhibit the same phenotype under a certain condition.

The term ‘heterologous protein production’ as used herein refers to the production of a protein which is not endogenous to the cell. It means an expression of a gene or part of a gene, particularly a transgene in a host CHO cell which does not naturally express this gene. The assays that are commonly used to quantify heterologous protein production include enzyme-linked immunosorbent assay (ELISA), chromatography & bioprocess analyser. The term ‘host cell’ as used herein refers to a cellular system for the expression of heterologous protein. For example, CHO cells are the main hosts for the production of various therapeutic proteins.

The term ‘optimal heterologous protein production’ herein refers to CHO cells that are capable of high-level protein production, particularly during industrial production or large-scale production of recombinant proteins, where the protein is usually a functional protein that is not naturally occurring in the wild-type CHO cell. In particular, for optimal heterologous protein production a CHO cell line has minimized metabolic burdens and toxic effects to the cell. More in particular, ‘optimal heterologous protein production’ refers to high level protein production where the CHO cell line not only produces a high yield of the protein of interest but also that the protein production is constantly maintained over the period of production (i.e., the prolonged period of culture) such that the quality of the protein produced is also consistent and maintained. In particular, for a CHO cell according to any aspect of the present invention to be capable of ‘optimal heterologous protein production’, the cell must at least display one of more of the following phenotypes of interest: phenotypic homogeneity, protein productivity, and protein quality. More in particular, for ‘optimal heterologous protein production’, the CHO cell may comprise phenotypic homogeneity and protein productivity, or phenotypic homogeneity, and protein quality, or protein productivity, and protein quality, or phenotypic homogeneity, protein productivity, and protein quality.

The term ‘protein productivity’ as used herein refers to a measure of the amount of protein made per viable cell at a single titer point. It is calculated by dividing the titer (mg) by the viable cell density (VCD or cells/ml), and the final measurement is represented as the amount of protein per cell (mg/cell).

The term ‘protein quality’ refers to the posttranslational modification of the protein that determines the efficacy and function of the protein. The modifications generally include phosphorylation, glycosylation, ubiquitination, methylation, acetylation, protein folding etc. For example, protein glycosylation is a critical quality attribute that modulates the efficacy, stability, and half-life of a therapeutic protein. Protein quality can be determined using Immunoprecipitation based techniques, Biochemical Assays, Mass spectrometry (MS) and the like.

The terms “methylation profile”, “methylation pattern”, “methylation state” or “methylation status,” are used herein to describe the state, situation or condition of methylation of a genomic sequence, and such terms refer to the characteristics of a DNA segment at a particular genomic locus in relation to methylation. Such characteristics include, but are not limited to, whether any of the cytosine (C) residues within this DNA sequence are methylated, location of methylated C residue(s), percentage of methylated C at any particular stretch of residues, and allelic differences in methylation due to, e.g., difference in the origin of the alleles.

The term “methylation status” refers to the status of a specific methylation site (i.e. methylated vs. non-methylated) which means a residue or methylation site is methylated or not methylated. Then, based on the methylation status of one or more methylation sites, a methylation profile may be determined. Accordingly, the term “methylation profile” or also “methylation pattern” refers to the relative or absolute concentration of methylated C residues or unmethylated C residues at any particular stretch of residues in the genomic material of a biological sample. For example, if cytosine (C) residue(s) not typically methylated within a DNA sequence are methylated, it may be referred to as “hypermethylated”; whereas if cytosine (C) residue(s) typically methylated within a DNA sequence are not methylated, it may be referred to as “hypomethylated”. Likewise, if the cytosine (C) residue(s) within a DNA sequence (e.g., the DNA from a sample nucleic acid from a test subject) are methylated as compared to another sequence from a different region or from a different individual (e.g., relative to normal nucleic acid or to the standard nucleic acid of the reference sequence), that sequence is considered hypermethylated compared to the other sequence. Alternatively, if the cytosine (C) residue(s) within a DNA sequence are not methylated as compared to another sequence from a different region or from a different individual, that sequence is considered hypomethylated compared to the other sequence. These sequences are said to be “differentially methylated”. Measurement of the levels of differential methylation may be done by a variety of ways known to those skilled in the art. One method is to measure the methylation level of individual interrogated CpG sites determined by the bisulfite sequencing method, as a non-limiting example.

The term “hypermethylation” refers to the average methylation state corresponding to an increased presence of 5-mCyt at one or a plurality of CpG dinucleotides within a DNA sequence of a test

DNA sample, relative to the amount of 5-mCyt found at corresponding CpG dinucleotides within a normal control DNA sample.

The term “hypomethylation” refers to the average methylation state corresponding to a decreased presence of 5-mCyt at one or a plurality of CpG dinucleotides within a DNA sequence of a test DNA sample, relative to the amount of 5-mCyt found at corresponding CpG dinucleotides within a normal control DNA sample.

As used herein, a “methylated nucleotide” or a “methylated nucleotide base” refers to the presence of a methyl moiety on a nucleotide base, where the methyl moiety is usually not present in a recognized typical nucleotide base. For example, cytosine in its usual form does not contain a methyl moiety on its pyrimidine ring, but 5-methylcytosine contains a methyl moiety at position 5 of its pyrimidine ring. Therefore, cytosine in its usual form may not be considered a methylated nucleotide and 5-methylcytosine may be considered a methylated nucleotide. In another example, thymine may contain a methyl moiety at position 5 of its pyrimidine ring, however, for purposes herein, thymine may not be considered a methylated nucleotide when present in DNA. Typical nucleotide bases for DNA are thymine, adenine, cytosine and guanine. Typical bases for RNA are uracil, adenine, cytosine and guanine. Correspondingly a “methylation site” is the location in the target gene nucleic acid region where methylation has the possibility of occurring. For example, a location containing CpG is a methylation site wherein the cytosine may or may not be methylated. In particular, the term “methylated nucleotide” refers to nucleotides that carry a methyl group attached to a position of a nucleotide that is accessible for methylation. These methylated nucleotides are usually found in nature and to date, methylated cytosine that occurs mostly in the context of the dinucleotide CpG, but also in the context of CpNpG- and CpNpN-sequences may be considered the most common. In principle, other naturally occurring nucleotides may also be methylated but they will not be taken into consideration with regard to any aspect of the present invention.

As used herein, the term “significantly similar” refers to in particular in context with the comparison of methylation profiles (such as the comparison between test profiles (from test subject(s) and reference profiles) a similarity observed by statistical means (i.e. by using bioinformatics) and/or also by observation using the eye. A significant similarity is observed for example if a test profile overlaps with a reference profile that is defined by multiple training samples through multivariate statistical methods, such as Principal Component analysis or Multi-Dimensional Scaling. In particular, a test profile is significantly similar to the pre-determined reference profile if more than 50, 55, 60, 65, 70, 75, 80, 85, 90, 95% of the methylation pattern/profile overlaps with that of the reference profile. A similarity of a test profile to more than one, such as two, three or even all reference profile reduces the significance of the similarity.

As used herein, the term “genomic material” refers to nucleic acid molecules or fragments of the genome of the CHO cells or cell lines. In particular, such nucleic acid molecules or fragments are DNA or RNA or hybrids thereof, and most preferably are molecules of the DNA genome of CHO cells or cell lines.

As used herein, the “DNA sample” refers to the DNA extracted from the cell according to any aspect of the present invention using known methods in the art.

‘Bisulfite treatment’ of genomic DNA used interchangeably with the term ‘bisulfite modification’, refers to the treatment of the genomic DNA with a deaminating agent such as a bisulfite that may be used to treat all DNA, methylated or not. In particular, the term “bisulfite” as used herein encompasses any suitable type of bisulfite, such as sodium bisulfite, or other chemical agents that are capable of chemically converting a cytosine (C) to an uracil (U) without chemically modifying a methylated cytosine and therefore can be used to differentially modify a DNA sequence based on the methylation status of the DNA, e.g., U.S. Pat. Pub. US 2010/0112595. As used herein, a reagent that “differentially modifies” methylated or non-methylated DNA encompasses any reagent that modifies methylated and/or unmethylated DNA in a process through which distinguishable products result from methylated and non-methylated DNA, thereby allowing the identification of the DNA methylation status. Such processes may include, but are not limited to, chemical reactions (such as a C to U conversion by bisulfite) and enzymatic treatment (such as cleavage by a methylation-dependent endonuclease). Thus, an enzyme that preferentially cleaves or digests methylated DNA is one capable of cleaving or digesting a DNA molecule at a much higher efficiency when the DNA is methylated, whereas an enzyme that preferentially cleaves or digests unmethylated DNA exhibits a significantly higher efficiency when the DNA is not methylated.

Accordingly, before step (a) according to any aspect of the present invention is carried out, the genomic DNA contained/obtained or extracted from the cell, is first bisulfite treated.

An alternative method available in the art may be used instead of bisulfite treatment. A skilled person will understand which other methods to use. In one example, TET-assisted pyridine borane sequencing (TAPS) may be used for detection of 5 mC and 5 hmC (Yibin Liu, et al., Nature Biotechnology, 37:424-429 (2019).

The term “test” used in conjunction with the term cell herein refers to a cell that is subjected to the method according to any aspect of the present invention and is the basis for an analysis application of the present invention. A ‘test cell’ is therefore a CHO cell or a group of CHO cells being tested according to any aspect of the present invention, or a profile being obtained or generated in this context. Conversely, the term “reference” or ‘control’ shall denote, mostly predetermined, entities which are used for a comparison with the test entity. In particular, a ‘test cell’ refers to a cell being tested for suitability of optimal homologous protein production where the methylation status has to be determined and a ‘control’ or ‘reference’ refers to a cell which is known to display optimal homologous protein production or a methylation profile thereof.

As used herein, a “CpG site” or “methylation site” is a nucleotide within a nucleic acid (DNA or RNA) that is susceptible to methylation either by natural occurring events in vivo or by an event instituted to chemically methylate the nucleotide in vitro. Some of these sites may be hypermethylated and some may be hypomethylated in a cell. In some cases a CpG site may not be considered fully hypermethylated or hypomethylated but a value may be given that is a measure of methylation of the CpG site. Accordingly, methylation may be quantified and may not always be an absolute case of hypermethylation or hypomethylation.

As used herein, a “methylated nucleic acid molecule” refers to a nucleic acid molecule that contains one or more nucleotides that is/are methylated.

A “CpG island” as used herein describes a segment of DNA sequence that comprises a functionally or structurally deviated CpG density. For example, Yamada et al. have described a set of standards for determining a CpG island: it must be at least 400 nucleotides in length, has a greater than 50% GC content, and an OCF/ECF ratio greater than 0.6 (Yamada et al., 2004, Genome Research, 14, 247-266). Others have defined a CpG island less stringently as a sequence at least 200 nucleotides in length, having a greater than 50% GC content, and an OCF/ECF ratio greater than 0.6 (Takai et al., 2002, Proc. Natl. Acad. Sci. USA, 99, 3740-3745).

In particular, when there is differential methylation detected in a test cell, that is to say that the cell displays absolute hypermethylation or hypomethylation or at least quantitative differential methylation at, at least one CpG site in comparison to the reference (i.e., from a CHO cell line with at least one phenotype of interest), then the test cell also comprises the phenotype of interest and may be capable of optimal heterologous protein production. More in particular, when the CpG site displays the same methylation status in the test cell in comparison to the corresponding CpG site in the reference cell or reference methylation profile, the test cell expresses the phenotype of interest and may be capable of optimal heterologous protein production. Overall, this platform gives us an opportunity to detect wide-spread DNA methylation status in CHO cells and correlate it with industrially relevant parameters which are crucial for the development of at least biological pharmaceutical products.

In particular, in the method according to any aspect of the present invention, in step (a) the methylation status of at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100 CpG sites are determined. A skilled person would be capable of determining the number of CpG sites that need to be used in step (a) according to any aspect of the present invention. Even more in particular, the methylation status of at least two CpG sites are determined in step (a) of the method according to any aspect of the present invention.

The term ‘epigenetic change’ as used herein refers to a chemical (e.g., methylation) change or protein (e.g., histones) change that takes place to a gene body or a promoter thereof. Through epigenetic changes, environmental factors like. diet, stress and prenatal nutrition can make an imprint on genes passed from one generation to the next.

In particular, the reference methylation profile according to any aspect of the present invention is a compilation of more than one CpG site from at least one CHO reference cell line that displays at least one phenotype of interest for optimal heterologous protein production. In one example, the different CpG sites are collected from a single reference CHO cell line that displays at least one phenotype of interest for optimal heterologous protein production. In another example, the different CpG sites are collected from more than one cell line where each cell line displays at least one phenotype of interest for optimal heterologous protein production. The reference methylation profile according to any aspect of the present invention may thus not be a naturally occurring methylation profile from a single CHO cell line but an artificial profile obtained from combining relevant CpG sites from different reference CHO cell lines, each with at least one phenotype of interest for optimal heterologous protein production.

The phenotype of interest for optimal heterologous protein may be selected from the group consisting of phenotypic homogeneity, protein productivity, and protein quality.

According to a further aspect of the present invention, there is provided a method of selecting at least one CHO cell comprising a phenotype of interest from a population of CHO cells from a parental clone, the method comprising the steps of:
(a) determining a test methylation profile from genomic material obtained from the CHO cell, and
(b) comparing the test methylation profile of (a) with a reference methylation profile from a parental clone displaying the phenotype of interest,
wherein a significant similarity between the test methylation profile and the reference methylation profile of (b) is indicative of the cell having the phenotype of interest of the parental clone; and
wherein the phenotype of interest is selected from the group consisting of phenotypic homogeneity, protein productivity, and protein quality and wherein the test methylation profile and reference methylation profile are from CpG sites from the CHO cell genome and are determined using DNA methylation-bead-based array.

As used herein, the term ‘parental clone’ refers to a cell line derived from host cells (a CHO cell line) in which a transgene has been integrated into the genome. The term ‘subclone’ as used herein in relation to a parental clone refers to a clonal cell line derived from parental clone having the same genotype but a different phenotype due to epigenetic changes.

The method used according to this aspect of the present invention is to select at least one CHO cell that is genetically and phenotypically identical or significantly similar to the parental clone in at least one bioreactor. In particular, usually during cell replication in a bioreactor of a parental clone, phenotypic plurality occurs. As used herein, the term ‘phenotypic plurality’ refers to a variation in phenotypes that exists within a cell population, particularly CHO cells, without any alteration of genotype under a certain specific condition. The method according to this aspect of the present invention allows for selecting at least one clone with least variation from the original/established parental clone that may also display a phenotype of interest (for example production of at least one human-like protein) out of phenotypically heterogenous population of CHO cells. In particular, by comparing distribution of CpG site methylation (e.g., beta value distribution) in the clonal population of a bioreactor, CHO cells that are identical or significantly similar to the parental clone may be identified. CHO cells that are identical or significantly similar to the parental CHO cell line may have the same methylation profile. Partially methylated clonal populations may also show cell-to-cell variation.

Similarly, the method used according to this aspect of the present invention is to select at least one CHO cell or a clonal population with selective and specific methylation profile for protein productivity. In this example, the selected CHO cells have the same methylation profile as the parental clone where the parental clone exhibits protein productivity. The reference methylation profile in this context thus refers to a methylation profile of the parental clone with protein productivity.

In another example, the method used according to this aspect of the present invention is to select at least one CHO cell or a clone population with selective and specific methylation profile for protein quality. Protein quality may be measured based on ideal glycosylation/sugar backbone and the like. In this example, the selected CHO cells have the same methylation profile as the parental clone where the parental clone exhibits protein quality. The reference methylation profile in this context thus refers to a methylation profile of the parental clone with protein quality.

According to a further aspect of the present invention, there is provided a method of identifying at least one CHO test cell line that is capable of producing at least one biosimilar relative to a heterologous protein produced by a CHO reference cell line, the method comprising the steps of:
(a) determining a test methylation profile from genomic material obtained from the CHO test cell line, and
(b) comparing the test methylation profile of (a) with the reference methylation profile of the CHO reference cell line,
   wherein a significant similarity between the test methylation profile of (a) and the reference methylation profile is indicative of the two cell lines producing biosimilars and wherein the test methylation profile and reference methylation profile are from CpG sites from the CHO cell genome and are determined using DNA methylation-bead-based array.

The term ‘biosimilar’ as used herein refers to recombinant proteins produced by genetically modified CHO cells which are highly similar to the original biotherapeutic reference product and share quality, safety and efficacy with the reference product. In particular, the product produced is phenotypically/epigenetically similar to the reference product. The term ‘biosimilar’ is more clearly explained at least in A. Ishii-Watabe, et al., (2019) Drug Metab. Pharmacokinet. 34 (1): 64-70 and Wolff-Holz, E., et al., (2019) BioDrugs 33, 621-634.

Information on DNA methylation patterns for cell lines could result in a clearer specification profile for product release in CHO cells and could serve as a “copyright” protection from biosimilar developers, and could develop as potential “gold standard”, for the regulatory process required for biosimilar development.

According to yet another aspect of the present invention, there is provided a method of A method of identifying at least one CHO test cell line that is capable of producing at least one bio-identical relative to a heterologous protein produced by a CHO reference cell line, the method comprising the steps of:
(a) determining a test methylation profile from genomic material obtained from the CHO test cell line, and
(b) comparing the test methylation profile of (a) with the reference methylation profile of the CHO reference cell line,
   wherein when the test methylation profile of (a) and the reference methylation profile are identical, it is indicative of the two cell lines producing bio-identicals and wherein the test methylation profile and reference methylation profile are from CpG sites from the CHO cell genome and are determined using DNA methylation-bead-based array.

As used herein, the term ‘bioidentical’ refers to recombinant proteins produced by genetically modified CHO cells that have the same molecular structure as the original biotherapeutic reference product. The term ‘bioidentical’ is more clearly explained at least in Stanczyk F Z, et al., Climacteric. 2021; 24:38-45.

CHO cells that are able to produce biosimilar or bioidentical proteins have a significantly similar or identical CpG methylation profile respectively to a reference profile from a CHO cell, particularly a parental clone that is capable of producing proteins most similar to the wildtype protein, particularly therapeutic protein. In another example, CHO cell that produce biosimilar or bioidentical proteins have a significantly similar or identical methylation profile of a selected region (e.g. but not restricted to low methylated regions (LMR)/partially methylated domains (PMD)/differentially methylated regions (DMR)/differentially methylated points (DMP) to a reference profile from a CHO cell, particularly a parental clone that is capable of producing proteins most similar to the wildtype protein, particularly therapeutic protein. In another example, the CHO cell that produce biosimilar or bioidentical proteins have a significantly higher CpG Methylation distribution (e.g., beta value distribution) compared to other CHO cells. In yet another example, a CHO cell that produce biosimilar or bioidentical proteins has no or the least amount of partial methylation at each site compared to other cells. In particular, the heterologous protein is a monoclonal antibody and/or therapeutic protein.

Low Methylated Region (LMR) is a region of the genome wherein less than 60% of CpGs in that region are methylated. More in particular, less than 50%, 40%, 30%, 20% or 10% of the CpGs in the LMRs are methylated. Any method known in the art may be used to identify or detect LMRs in the genomic DNA. Well known methods include using programmes such as MethylSeekR. In particular, LMRs in the genomic DNA have at least three consecutive CpGs and have no single nucleotide polymorphisms (SNPs) in any of the CpG positions. Even more in particular, LMRs in the genomic DNA are identified based on the method disclosed at least in Burger, L., (2013) Nucleic Acids Research, 41 (16): e155 and/or Stadler, M., (2011) Nature 480, 490-495. LMRs are known to have an average methylation ranging from 10% to 50%; are regions of low CG density which do not overlap with CpG islands; tend to be enriched for H3K4me1, DHSs, and p300/CBP; and/or are primarily located distal to promoters in intergenic or intronic regions. In particular, LMRs:
have an average methylation ranging from 10% to 50%,
are regions of low CG density;
are enriched for Histone H3 monomethylated at lysine 4 (H3K4me1), DNase I hypersensitive sites (DHSs) and transcriptional coactivators CREB binding protein (CPB) and p300;
are primarily located distal to promoters in intergenic or intronic regions; and/or
have no single nucleotide polymorphisms (SNPs) in any of the CpG positions.

Low-methylated regions (LMRs) represent a key feature of the dynamic methylome. LMRs are local reductions in the DNA methylation landscape and represent CpG-poor distal regulatory regions that often reflect the binding of transcription factors and other DNA-binding proteins. LMRs were originally described in the mouse (Stadler et al. (2011) Nature: 480, 490-95). Evolutionary conservation of LMRs beyond mammals has remained unexplored.

Differentially methylated regions (DMRs) are genomic regions with different methylation statuses among multiple biological samples like tissues, cells, individuals, etc. These are genomic regions that differ between phenotypes. The statistical power is likely to be greater when adjacent DMPs are considered together as a whole [Gu H et al (2010) Nat Methods 2010; 7:133-6]. The lengths of the DMRs may range between a few hundred to a few thousand bases [Rakyan et al (2011) Nat Rev Genet 12:529-41, 2011, Bock C (2012) Nat Rev Genet 2012; 13:705-19].

DMRs may occur throughout the genome but have been identified particularly around the promoter regions of genes, within the body of genes, and at intergenic regulatory regions. There are two types of regions, predefined or user defined. Regions with special biological meaning, such as CpG islands, CpG shores, UTRs and so on, are predefined. Many traditional statistical testings, including t-test and Wilcoxon rank sum test, can be performed at a region level. For user-defined regions, criteria such as a fixed region length, fixed numbers of significant and adjacent CpG sites, significant and smoothed estimated effect sizes, etc.

Partially methylated domains (PMDs) are extended regions in the genome exhibiting a reduced average DNA methylation level. They cover gene-poor and transcriptionally inactive regions and tend to be heterochromatic.

Differentially methylated Positions (DMP) are CpG sites with different DNA methylation status across different biological samples and regarded as possible functional regions involved in gene transcriptional regulation.

According to a further aspect of the present invention, there is provided a method for assessing one or more phenotypic parameters of at least one test CHO cell line, the method comprising the steps of
(a) determining a test methylation status of one or more pre-selected methylation sites from the genomic material obtained from the test CHO cell line;
(b) determining from the methylation status determined in (a) a test methylation profile of the test CHO cell line; and
(c) comparing the test methylation profile determined in (b) with at least one predetermined reference methylation profiles, wherein each of the predetermined reference methylation profiles is specific for a reference CHO cell line with at least one phenotypic parameters;
wherein if the test methylation profile is significantly similar to one of the predetermined reference methylation profiles, the test CHO cell line has similar, or preferably the same phenotypic parameter as the reference CHO cell line with the predetermined reference methylation profile and wherein the test methylation profile and reference methylation profiles are from CpG sites from the CHO cell genome and are determined using DNA methylation-bead-based array.

In particular, the phenotypic parameter is selected from the group consisting of:
Optimal carbohydrate metabolism
Optimal amino acid metabolism
Optimal lipid metabolism
Optimal protein productivity; and
Optimal cell survivability

The term ‘carbohydrate metabolism’, as used herein refers to almost all or all of the biochemical processes responsible for the metabolic formation, breakdown, and interconversion of carbohydrates in cells. It involves multiple pathways such as glycolysis, gluconeogenesis, glycogenolysis, and glycogenesis. For example, glycolysis is one of the key metabolic pathways of CHO cells. Through glycolysis, CHO cells consume glucose as the main carbon source for energy production and generate lactate as the most common metabolic by-product. Particularly, the term ‘optimal carbohydrate metabolism’ refers to the ideal or best carbohydrate metabolism possible by a CHO cell.

Similarly, the term ‘amino acid metabolism’ as used herein refer to the whole of the biochemical processes responsible for the metabolic formation, breakdown, and interconversion of amino acids in cells. Amino acids are the basic building blocks of proteins and constitute all proteinaceous material of the cell including the cytoskeleton, protein component of enzymes, receptors, and signalling molecules. In addition, amino acids are utilized for the growth and maintenance of cells. For example, glutaminolysis is a key metabolic pathway of CHO cells. Glutaminolysis is the prevalent pathway through which CHO cells assimilate organic nitrogen for biomass synthesis while releasing ammonium as the main by-product. Particularly, the term ‘optimal amino acid metabolism’ refers to the ideal or best amino acid metabolism possible by a CHO cell.

The term ‘lipid metabolism’ as used herein refers to the synthesis and degradation of lipids in cells, involving the breakdown or storage of fats for energy and the synthesis of structural and functional lipids. Lipids are the major component of cellular membranes, act as secondary messengers in cell communication, involved in signalling, transport and secretion. Lipids are also an important source of energy through B-oxidation and the tricarboxylic acid (TCA) cycle. Lipid metabolism can have a significant impact on cell growth. For example, the process of triacylglycerol synthesis and degradation in CHO cells can greatly affect overall cellular metabolism and viability. Particularly, the term ‘optimal lipid metabolism’ refers to the ideal or best amino acid metabolism possible by a CHO cell.

Carbohydrate, amino acid and lipid metabolism can be determined by Metabolite Detection Assays, HPLC and bioprocess analyser. These methods are further disclosed at least in Coulet, M. et al., Cells (2022), 11, 1929; Fan Y, et al., Biotechnol Bioeng (2015) 112(3):521-535 and Ali A S, et al., Biotechnol J. (2018); 13(10):e1700745.

As used herein, the term “pre-selected methylation sites” refers to methylation sites that were selected from genes or regions that showed the highest degree of methylation variation during the training of the method and fulfils certain quality criteria such as a minimum sequencing coverage of ≥5× were considered and for ≥5 qualified CpG sites. Additionally, genes that have an average methylation level <0.1 or an average methylation level >0.9 can be excluded due to their limited dynamic range. “Reference methylation profiles” may be defined on the basis of multiple training samples using multivariate statistical methods, such as such as Principal Component analysis or Multi-Dimensional Scaling.

The term “significantly similar” as used herein, and in particular in context with the comparison of methylation profiles (such as the comparison between test profiles (from test subject(s) and reference profiles) shall mean a similarity observed by statistical means (i.e. by using bioinformatics) and/or also by observation using the eye. A significant similarity is observed for example if a test profile overlaps with a reference profile that is defined by multiple training samples through multivariate statistical methods, such as Principal Component analysis or Multi-Dimensional Scaling. In particular, a test profile is significantly similar to the pre-determined reference profile if more than 50, 55, 60, 65, 70, 75, 80, 85, 90, 95% of the methylation pattern/profile overlaps with that of the reference profile. A similarity of a test profile to more than one, such as two, three or even all reference profiles reduce the significance of the similarity. The term “pre-determined reference profile” as used herein refers to a typical or standard methylation profile of the genomic material of a CHO cell line with a specific feature dependent on the context where the term is used. In one example, for a method of determining a CHO cell line that displays at least one phenotypic parameter according to any aspect of the present invention conferring the potential of optimal heterologous protein production on the cell line, the term “pre-determined reference profile” refers to a typical or standard methylation profile of the genomic material of the CHO cell line displaying one or more of the phenotypic parameters selected from the group consisting of optimal glucose consumption, optimal growth rate, optimal lactic acid production, and optimal ammonia accumulation. The pre-determined reference profile may be obtained from one or more reference CHO cell lines each expressing one or more phenotypic parameter.

The method according to this aspect of the present invention attempts to create a methylation profile for a CHO cell line that has the potential for optimal heterologous protein production as the cell line may exhibit cell survivability, fitness, low cell exhaustion and good metabolic readouts. In particular, the method according to this aspect of the present invention provides a prognostic methylation profile for ideal parental cell lines prior to transgene introduction.

According to yet another aspect of the present invention, there is provided a method for developing a test system for determining if a test CHO cell line is capable of optimal heterologous protein production, the method comprising the steps of:
(a) determining a test methylation status of one or more pre-selected methylation sites from the genomic material obtained from the test CHO cell line;
(b) selecting from the pre-selected methylation sites a reference panel of methylation sites which is characterized by a specific and distinct differential methylation profile for each phenotypic parameter or phenotype of interest;
(c) obtaining a test system by assigning a reference methylation profile for each of the phenotypic parameter or phenotypes of interest; and
wherein a comparison of a test methylation profile obtained from a test sample with the reference methylation profiles obtained in (c) allows for confirming if the test CHO cell line is capable of optimal heterologous protein production and wherein the test methylation profile and reference methylation profiles are from CpG sites from the CHO cell genome and are determined using DNA methylation-bead-based array.

The term ‘a reference panel of methylation sites’ refers to specific and distinct CpG sites or regions that are used to form the reference methylation profile.

According to yet another aspect of the present invention, there is provided a method of determining if a CHO cell line is robust, stable and capable of optimal heterologous protein production before introduction of a transgene into the cell, the method comprising the steps of:
(a) determining a methylation profile from genomic material obtained from the CHO cell line; and
(b) comparing the methylation profile of (a), with a reference methylation profile for a CHO cell line that is robust, stable and capable of optimal heterologous protein production,
wherein a significant similarity between the test methylation profile of (a) and the reference methylation profile is indicative of the CHO cell line being robust, stable and capable of optimal heterologous protein production and wherein the test methylation profile and reference methylation profile are from CpG sites from the CHO cell genome and are determined using DNA methylation-bead-based array.

The DNA methylation profile of step (a) according to any aspect of the present invention is determined using DNA methylation-based array. In particular, a bead-based DNA methylation array. The array according to any aspect of the present invention is advantageous as it enables the understanding of genome stability of the CHO cell line, enables better control over the manufacturing/process development/product development/scaling up/validation process, thereby aiding in the selection of better CHO cell lines for industrial applications.

DNA-Methylation-based arrays allow for a high-throughput and robust method to determine semi-quantitative/quantitative DNA-methylation information through a small sample of extracted DNA of interest. These custom designed arrays may use Illumina iScan and Infinium platform technology or an equivalent thereof, which allows on each chip for example 100,000 different bead types that covalently bind DNA-methylation probes. Each probe represents one CpG Methylation site at the end of the probe sequence. DNA samples undergo bisulfite conversion, amplification, fragmentation, precipitation and resuspension steps before hybridization on an array chip. Once on the chip the DNA hybridizes to the beads for each CpG site so that methylation changes at each site can be detected specifically through single nucleotide extension. This is especially advantageous as the array-based method is simple and the results of the methylation-based array are accurate and reproducible.

Further, compared to traditional sequencing which can take weeks to generate data, the array technology has a much shorter turn-around time. The volume and complexity of data generated is lesser compared to sequencing making it computationally less intensive. This allows for quicker computation to achieve interpretable results from experimental groups. Overall microarray technology is roughly 10x faster and 10x cheaper than traditional sequencing while still quantifiable for the methylation level at specific CpG sites.

The term “array” as used herein refers to an intentionally created collection of probe molecules which can be prepared either synthetically or biosynthetically. The probe molecules in the array can be identical or different from each other. The array can assume a variety of formats, for example, libraries of soluble molecules; libraries of compounds tethered to resin beads, silica chips, or other solid supports.

In particular, a DNA methylation-based array provides a convenient platform for simultaneous analysis of large numbers of CpG sites, for example, at least 2, 3, 4, 5, 6, 7, 8, 9, 10, 50, 100, 500, 1000, 5000, 10,000, 100,000 or more sites or loci. In particular, the array comprises a plurality of different probe molecules that can be attached to a substrate or otherwise spatially distinguished in an array. Examples of arrays that may be used according to any aspect of the present invention include slide arrays, silicon wafer arrays, liquid arrays, bead-based arrays and the like. In one example, array technology used according to any aspect of the present invention combines a miniaturized array platform, a high level of assay multiplexing, and scalable automation for sample handling and data processing.

In particular, the array according to any aspect of the present invention may be an array of arrays, also referred to as a composite array, having a plurality of individual arrays that is configured to allow processing of multiple samples simultaneously. Examples of composite arrays and the technology behind them are disclosed at least in U.S. Pat. No. 6,429,027 and US 2002/0102578. A substrate of a composite array may include a plurality of individual array locations, each having a plurality of probes, and each physically separated from other assay locations on the same substrate such that a fluid contacting one array location is prevented from contacting another array location. Each array location can have a plurality of different probe molecules that are directly attached to the substrate or that are attached to the substrate via rigid particles in wells (also referred to herein as beads in wells).

In one example, an array substrate can be a fibre optical bundle or array of bundles as described in U.S. Pat. Nos. 6,023,540, 6,200,737 and/or 6,327,410. An optical fibre bundle or array of bundles can have probes attached directly to the fibres or via beads. A skilled person would be able to easily determine which substrate will be most suitable for the array according to any aspect of the present invention. WO2004110246 further discloses other substrates and methods of attaching beads to the substrates that may be used in the array according to any aspect of the present invention.

In one example, a surface of the substrate may have physical alterations to enable the attachment of probes or produce array locations. For example, the surface of a substrate can be modified to contain chemically modified sites that are useful for attaching, either-covalently or non-covalently, probe molecules or particles having attached probe molecules. Probes may be attached using any of a variety of methods known in the art including, an ink-jet printing method, a spotting technique, a photolithographic synthesis method, or printing method utilizing a mask. WO2004110246 discloses these techniques in more detail.

In one example, the DNA methylation-based array according to any aspect of the present invention may be a bead-based array, where the beads are associated with a solid support such as those commercially available from Illumina, Inc. (San Diego, Calif.). An array of beads useful according to any aspect of the present invention can also be in a fluid format such as a fluid stream of a flow cytometer or similar device. Commercially available fluid formats for distinguishing beads include, for example, those used in XMAP™ technologies from Luminex or MPSS™ methods from Lynx Therapeutics.

The term “solid support”, “support”, and “substrate” as used herein are used interchangeably and refer to a material or group of materials having a rigid or semi-rigid surface or surfaces. In many examples, at least one surface of the solid support will be substantially flat, although in some examples it may be desirable to physically separate synthesis regions for different compounds with, for example, wells, raised regions, pins, etched trenches, or the like.

The DNA methylation array according to any aspect of the present invention may be a very high-density array, for example, those having from about 10,000,000 probes/cm² to about 2,000,000,000 probes/cm² or from about 100,000,000 probes/cm² to about 1,000,000,000 probes/cm². High density arrays are especially useful according to any aspect of the present invention for including the multitude of CpG sites on the array.

The DNA methylation array according to any aspect of the present invention may be used to analyse or evaluate such pluralities of loci simultaneously or sequentially as desired. In one example, a plurality of different probe molecules can be attached to a substrate or otherwise spatially distinguished in an array. Each probe is typically specific for a particular locus and can be used to distinguish methylation state of the locus.

The term “probe molecules” as used herein refers to a surface-immobilized molecule that can be recognized by a particular target. Probes used in the array can be specific for the methylated allele of a CpG site, the non-methylated allele of the CpG site or both.

The term “target” as used herein refers to a molecule that has an affinity for a given probe molecule. Targets may be naturally occurring or man-made molecules. Also, they can be employed in their unaltered state or as aggregates. Targets may be attached, covalently or noncovalently, to a binding member, either directly or via a specific binding substance. Examples of targets which can be employed according to any aspect of the present invention are methylated and non-methylated CpG sites. Targets are sometimes referred to in the art as anti-probes. As the term targets is used herein, no difference in meaning is intended.

The term “complementary” as used herein refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Perfectly complementary refers to 100% complementarity over the length of a sequence. For example, a 25-base probe is perfectly complementary to a target when all 25 bases of the probe are complementary to a contiguous 25 base sequence of the target with no mismatches between the probe and the target over the length of the probe.

According to another aspect of the present invention, there is provided a method of determining regulation of transgene expression in at least one CHO cell line genetically modified with the transgene, the method comprising the step of:
measuring the methylation level of at least one CpG site of at least one promoter of the transgene,
   wherein the promoters is a viral promoter; and
   wherein the DNA methylation level is determined using DNA methylation bead-based array.

According to another aspect of the present invention, there is provided a method of determining regulation of transgene expression in at least one CHO cell line genetically modified with the transgene, the method comprising the step of:
measuring the methylation level of at least one CpG site of at least one promoter of the transgene,
   wherein the promoters are selected from Cytomegalovirus (CMV) and simian vacuolating virus 40 (SV40); and
   wherein the DNA methylation level is determined using DNA methylation bead-based array.

As used herein, the terms “promoter” or “gene promoter” used interchangeably with the terms ‘regulatory region’ or ‘regulatory sequence’ refers to the respective contiguous gene DNA sequence extending from 1.5 kb upstream to 1.5 kb downstream relative to the transcription start site (TSS), or contiguous portions thereof. In particular, ‘regulatory region’ refers to the respective contiguous gene DNA sequence extending from 1.5 kb upstream to 0.5 kb downstream relative to the TSS. In some examples, ‘regulatory region’ refers to the respective contiguous gene DNA sequence extending from 1.5 kb upstream to the downstream edge of a CpG island that overlaps with the region from 1.5 kb upstream to 1.5 kb downstream from TSS (and is such cases, my thus extend even further beyond 1.5 kb downstream), and contiguous portions thereof. Change in DNA methylation on the gene promoters responsible for protein glycosylation can lead to an improvement of protein quality. Protein glycosylation is a critical quality attribute that modulate the efficacy, stability, and half-life of a therapeutic protein. It is desirable to obtain a consistent glycoform profile in protein production due to regulatory concerns. Hence, DNA methylation can act as a tool to globally regulate CHO metabolism and protein production.

According to a further aspect of the present invention, there is provided a use of DNA methylation profiling for identifying at least one suitable insertion site or region in genome of a CHO cell line for introduction of at least one transgene. In particular, with the information on CHO epigenome, suitable transgene insertion sites based on methylation patterns which are optimal ‘hot spots’ for transgene expression can be identified. For example, specific LMRs may be identified in the genome of the CHO cell line for a targeted insertion of at least one transgene for example as highly methylated sites would be silenced and not as productive for expression of transgenes (TIS analysis). In another example, CMV promoters and surrounding repetitive elements may be identified also as a hot spot for transgene insertion using methylation profiling.

Methylation profiling may also be used to screen and select suitable promotors for use in CHO cells that result in optimal transgene expression. In particular, methylation data from different promoters and transgene insertion sites may be obtained and compared to select the best performing promoters which can lead to improved transgene expression. In particular, the array according to any aspect of the present invention may be used to monitor activity of transgene (expression or silencing/imprinting) by quantifying the DNA methylation level of the transgene promoter According to yet another aspect of the present invention, there is provided a DNA methylation-bead based array comprising at least:
a plurality of distinct locations, each location having at least one probe molecule comprising a nucleic acid sequence complementary to a plurality of CpG sites of a CHO cell,
   wherein the CpG sites of the CHO cell are from the CHO genome and may be selected from at least one CpG in the Table 5a-5f.

These CpG sites are environment specific CpG sites (i.e. dynamic CpG sites), and CpG sites found in promoters and the genes per se of metabolic linked genes, protein production linked genes, cell growth and division linked genes, and epigenetic linked genes.

‘Environmental specific CpG sites’ also known as dynamic CpG sites in the context of CHO cells refer to the CpG sites that are differentially methylated among different CHO cell lines. The cell lines that were used in this analysis include CHO-K1 (ATCC), CHO-DG44 (Thermo Fisher Scientific), CHO-DXB11 (ATCC), ExpiCHO-S™ cells (Thermo Fisher Scientific), FreeStyle™ CHO-S™ cells (Thermo Fisher Scientific), CHO 1-15₅₀₀ (ATCC) and Agarabi CHO (ATCC).

‘Metabolic linked genes’ in the context of CHO cells herein refer to genes that are related to several metabolism pathways such as Glycolysis, TCA cycle, Pentose Phosphate pathway, Malate-aspartate shuttle, Amino acid metabolism, Lactate metabolism, Cholesterol biosynthesis, Nucleotide biosynthesis, Nucleotide sugar biosynthesis etc. A few examples of such genes include Hk2, Pgk1, Idh3a, Pgm1, and Pdha1. A skilled person would easily determine the genes that are found in CHO cells that fall within this category.

‘Protein production linked genes’ used in the context of CHO cells herein refer to genes that are related to cellular processes such as DNA replication and repair, mRNA transcription, mRNA translation, post-translational modifications, and protein folding and export. A few examples of such genes include Gatb, Sec61a2, Ube2e3, Exosc1, Dna2, Pold1 and the like. A skilled person would easily be able to determine the other genes that are found in CHO cells that fall within this category.

‘Cell growth and division linked genes’ used in the context of CHO cells herein refer to genes that are related to cellular processes such as cell cycle regulation, Cytoskeleton-related elements, cell signalling, nucleotide metabolism, and cell death. A few examples of such genes include Camk1, Cd82, Cdk4, Col1a1, and Ctsb. Again, a skilled person would easily be able to determine the other genes that are found in CHO cells that fall within this category.

‘Epigenetic linked genes’ used in the context of CHO cells herein refer to genes that are related to epigenetic modifications such as DNA methylation pathway, DNA demethylation pathway, Folate and Methionine cycle, and Histone modifications. A few examples of such genes include Hat1, Shmt1, Bhmt, Dnmt1, and Ehmt1. A skilled person would easily be able to determine the other genes that are found in CHO cells that fall within this category.

The term ‘Viral promoters’ used in the context of CHO cells herein refer to promoter and enhancer of at least the cytomegalovirus (CMV) and simian vacuolating virus 40. The viral promoters are usually rich in CpG sites which make them more prone to DNA methylation and thus suppressing the protein expression.

The methods according to any aspect of the present invention may also be used to predict if a CHO test cell is capable of optimal heterologous protein production.

### EXAMPLES

The foregoing describes preferred embodiments, which, as will be understood by those skilled in the art, may be subject to variations or modifications in design, construction or operation without departing from the scope of the claims. These variations, for instance, are intended to be covered by the scope of the claims.

### Example 1

### Oxidative Stress in CHO Cell Culture

### Wet-Lab Methodology

For this experiment, a transgenic CHO cell line, Agarabi CHO (ATCC® CRL-3440™), was grown in CD FortiCHO medium supplemented with 8 mM L-glutamine at 37° C., 8% CO2, at a shaking speed of 130 RPM. Batch culture of 6 flasks was maintained for 7 days where 3 flasks represent technical replicates for the control set and 3 flasks represent technical replicates for the treatment set. The flasks were seeded with 3E5 viable cells/mL on day 0 and to induce oxidative stress, hydrogen peroxide was added every 48 hrs to the treatment set with a final concentration of 120 μM. Cell count, cell viability, and heterologous protein production were measured every 2 days and cell pellets were collected for both control and treatment set on day 7. Induction of oxidative stress in CHO cells by treatment with hydrogen peroxide resulted in reduced growth rate and cell viability compared to control set and thus there was a slight increase in heterologous protein productivity for treatment set.

Genomic DNA was purified from the collected cell pellets using DNeasy Blood & Tissue Kit (Qiagen) and was quantified using PicroGreen or NanoDrop™ 2000. The genomic DNA (500 ng) from the control and treatment set were used to prepare libraries for Whole Genome Bisulfite Sequencing (WGBS). The sequencing of the libraries was performed by a third party on a NovaSeq platform which generated 125 GB of data per sample.

### Computational Methodology

Raw sequencing data were conducted quality control (fastqc) 1, sequencing adaptors trimming (TrimGalore)2, and alignment with Bismark3. CMV promoter combined with CHOK1-GS (Cricetulus griseus) genome was used as a reference genome. Bismark was also used for removing duplicated reads and extracting methylation counts from alignment output. SNPs were filtered out, and only counts with a minimum coverage of 10x were used for the downstream analysis, which resulted in 3711013 CpG sites for hydrogen peroxide treatment samples. Since regulated methylation targets are most commonly clustered into short regions, DMRfinder4 was used to perform a modified single-linkage clustering of methylation sites. With a maximum distance between CpG sites of 100 bp, 1728014 genomic regions were found for hydrogen peroxide treatment samples.

### Differential Methylation Analysis

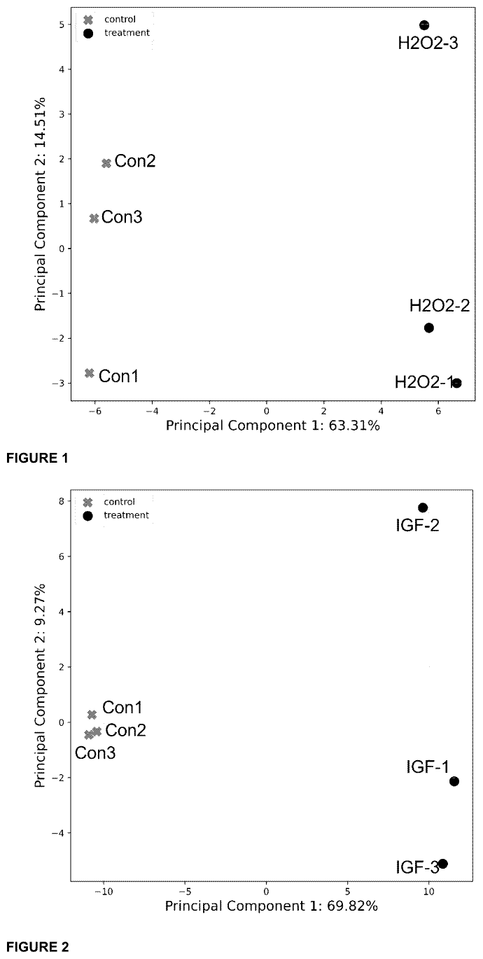

Differential methylation analysis was performed using MethylKit5 between the control and treatment groups. Logistic regression was used to determine the differential methylation across all regions, and the sliding linear model (SLIM) 6 method to do FDR correction. Regions with FDR corrected p-value <0.05 and methylation change greater than 25% between groups were determined as differentially methylated regions (DMRs), which were 122 for hydrogen peroxide treatment samples, shown in Table 1. Principal Component Analysis (PCA) is a dimensionality reduction technique that emphasizes variation in a dataset. PCA analysis for DMRs is shown in FIG. 1.

Preliminary results show DMRs play roles in epigenetic changes of oxidative stress, which can be potentially used as markers for future research.

TABLE 1
List of differentially methylated regions (DMRs)
identified in hydrogen peroxide treatment samples

| chr | start | end | chr | start | end |
|---|---|---|---|---|---|
| scaffold_11 | 28709706 | 28710166 | scaffold_17 | 22318231 | 22318310 |
| scaffold_37 | 2633223 | 2633699 | scaffold_2 | 64964306 | 64964784 |
| scaffold_3 | 51648552 | 51648916 | scaffold_27 | 2573631 | 2573958 |
| scaffold_5 | 69834725 | 69835199 | scaffold_0 | 126101885 | 126101986 |
| scaffold_8 | 5552117 | 5552561 | scaffold_27 | 7559618 | 7559924 |
| scaffold_26 | 21297189 | 21297609 | scaffold_0 | 141397264 | 141397482 |
| scaffold_3 | 89611520 | 89611822 | scaffold_15 | 11750842 | 11751221 |
| scaffold_22 | 26009928 | 26010211 | scaffold_18 | 18899911 | 18900021 |
| scaffold_5 | 71575709 | 71576056 | scaffold_31 | 11436108 | 11436414 |
| scaffold_1 | 39368692 | 39369030 | scaffold_22 | 11319316 | 11319522 |
| scaffold_5 | 18517703 | 18518011 | scaffold_10 | 7547985 | 7548213 |
| scaffold_29 | 14434642 | 14434837 | scaffold_29 | 19957756 | 19958066 |
| scaffold_30 | 17539176 | 17539422 | scaffold_13 | 1557582 | 1557813 |
| scaffold_35 | 1506683 | 1507183 | scaffold_31 | 216291 | 216630 |
| scaffold_2 | 33171492 | 33171654 | scaffold_18 | 1845104 | 1845449 |
| scaffold_29 | 21214834 | 21215287 | scaffold_6 | 47899988 | 47900353 |
| scaffold_31 | 7044527 | 7044866 | scaffold_2 | 3574161 | 3574486 |
| scaffold_4 | 36086102 | 36086595 | scaffold_22 | 19696673 | 19696913 |
| scaffold_67 | 1370390 | 1370856 | scaffold_2 | 27271033 | 27271300 |
| scaffold_35 | 11121893 | 11122057 | scaffold_48 | 589774 | 590045 |
| scaffold_38 | 12852538 | 12852906 | scaffold_3 | 54596703 | 54597029 |
| scaffold_1 | 39315830 | 39316127 | scaffold_0 | 119459381 | 119459708 |
| scaffold_0 | 34089755 | 34090116 | scaffold_22 | 557058 | 557163 |
| scaffold_6 | 72951899 | 72952171 | scaffold_17 | 20092773 | 20093109 |
| scaffold_92 | 551040 | 551276 | scaffold_0 | 175572250 | 175572386 |
| scaffold_12 | 35346308 | 35346549 | scaffold_27 | 4544681 | 4544868 |
| scaffold_7 | 66793870 | 66793964 | scaffold_3 | 35596025 | 35596105 |
| scaffold_8 | 5881102 | 5881479 | scaffold_38 | 7029674 | 7029838 |
| scaffold_5 | 27353205 | 27353427 | scaffold_16 | 6569320 | 6569338 |
| scaffold_6 | 38750584 | 38750737 | scaffold_31 | 13557315 | 13557751 |
| scaffold_19 | 29152850 | 29152892 | scaffold_22 | 27111725 | 27111786 |
| scaffold_10 | 44249680 | 44249959 | scaffold_24 | 31162074 | 31162369 |
| scaffold_31 | 19208325 | 19208599 | scaffold_2 | 23082378 | 23082645 |
| scaffold_0 | 89742694 | 89743065 | scaffold_0 | 172352541 | 172352799 |
| scaffold_22 | 10120022 | 10120299 | scaffold_3 | 117557801 | 117558116 |
| scaffold_5 | 75352416 | 75352711 | scaffold_1 | 53974390 | 53974471 |

| chr | start | end | chr | start | end |
|---|---|---|---|---|---|
| scaffold_31 | 17314786 | 17315000 | scaffold_32 | 10370015 | 10370095 |
| scaffold_31 | 15964682 | 15964838 | scaffold_2 | 23744711 | 23744782 |
| scaffold_3 | 25998505 | 25998572 | | | |
| scaffold_35 | 13279321 | 13279421 | | | |
| scaffold_9 | 14408479 | 14408926 | | | |
| scaffold_9 | 22223538 | 22223924 | | | |
| scaffold_2 | 15803480 | 15803773 | | | |
| scaffold_22 | 31474980 | 31475143 | | | |
| scaffold_100 | 220811 | 221203 | | | |
| scaffold_0 | 28634272 | 28634445 | | | |
| scaffold_6 | 65899215 | 65899457 | | | |
| scaffold_2 | 90082675 | 90082850 | | | |
| scaffold_2 | 45648573 | 45648704 | | | |
| scaffold_10 | 46239592 | 46239793 | | | |
| scaffold_31 | 17811929 | 17812041 | | | |
| scaffold_22 | 3209376 | 3209491 | | | |
| scaffold_33 | 2624404 | 2624623 | | | |
| scaffold_0 | 220810084 | 220810236 | | | |
| scaffold_45 | 4457534 | 4457636 | | | |
| scaffold_0 | 19094594 | 19094694 | | | |
| scaffold_7 | 15516433 | 15516528 | | | |
| scaffold_3 | 38128986 | 38129133 | | | |
| scaffold_12 | 28364487 | 28364704 | | | |
| scaffold_34 | 4301026 | 4301187 | | | |
| scaffold_0 | 187893529 | 87893812 | | | |
| scaffold_5 | 8164287 | 8164373 | | | |
| scaffold_1 | 102361970 | 102362116 | | | |
| scaffold_3 | 6951744 | 6951853 | | | |
| scaffold_2 | 13527644 | 13528097 | | | |
| scaffold_8 | 35652365 | 35652442 | | | |
| scaffold_3 | 27019769 | 27019916 | | | |
| scaffold_35 | 281510 | 281564 | | | |
| scaffold_29 | 26268335 | 26268472 | | | |
| scaffold_13 | 41328125 | 41328356 | | | |
| scaffold_61 | 1889662 | 1889727 | | | |
| scaffold_0 | 147163257 | 147163431 | | | |

### Example 2

Adaptation of CHO Cells with Media Supplements

### Wet-Lab Methodology

For this experiment, a transgenic CHO cell line, Agarabi CHO (ATCC® CRL-3440™), was adapted for 2 weeks in CD FortiCHO medium supplemented with 8 mM L-glutamine & 1 mg/L human insulin-like growth factor 1 (IGF-1) at 37° C., 8% CO2, at a shaking speed of 130 RPM. Batch culture of 6 flasks was maintained for 7 days where 3 flasks represent technical replicates for the control set (without IGF-1 adaptation) and 3 flasks represent technical replicates for the IGF-1 adapted set. The flasks were seeded with 3E5 viable cells/mL on day 0 and 1 mg/L Insulin Growth Factor was added to the adapted set. Cell count, cell viability, and protein production were measured every 2 days and cell pellets were collected for both control and treatment set on day 7. Adaptation of CHO cells with IGF-1 had no significant effect on growth rate and viability, however, heterologous protein productivity was doubled as compared to the control set.

Genomic DNA was purified from the collected cell pellets using DNeasy Blood & Tissue Kit (Qiagen) and was quantified using PicroGreen or NanoDrop™ 2000. The genomic DNA (500 ng) from the control and adapted set were used to prepare libraries for Whole Genome Bisulfite Sequencing (WGBS). The sequencing of the libraries was performed by a third party on a NovaSeq platform which generated 125 GB of data per sample.

### Computational Methodology

Raw sequencing data were conducted quality control (fastqc) 1, sequencing adaptors trimming (TrimGalore) 2, and alignment with Bismark3. CMV promoter combined with CHOK1-GS (Cricetulus griseus) genome was used as a reference genome. Bismark was also used for removing duplicated reads and extracting methylation counts from alignment output. SNPs were filtered out, and only counts with a minimum coverage of 10x were used for the downstream analysis, which resulted in 4244091 CpG sites for IGF-1 adapted samples. Since regulated methylation targets are most commonly clustered into short regions, DMRfinder4 was used to perform a modified single-linkage clustering of methylation sites. With a maximum distance between CpG sites of 100 bp, 2048904 genomic regions were found for IGF-1 adapted samples.

### Differential Methylation Analysis

Differential methylation analysis was performed using MethylKit5 between the control and adapted groups. Logistic regression was used to determine the differential methylation across all regions, and the sliding linear model (SLIM) 6 method to do FDR correction. Regions with FDR corrected p-value <0.05 and methylation change greater than 25% between groups were determined as differentially methylated regions (DMRs), which was 289 for IGF-1 adapted samples listed in Table 2. Principal Component Analysis (PCA) is a dimensionality reduction technique that emphasizes variation in a dataset. PCA analysis for DMRs is shown in FIG. 2.

Preliminary results show DMRs play roles in epigenetic changes of IGF-1 adaptation, which can be potentially used as markers for future research.

TABLE 2a
List of differentially methylated regions
(DMRs) identified in IGF-1 adapted samples.

| chr | start | end | chr | start | end |
|---|---|---|---|---|---|
| scaffold_8 | 14408898 | 14409225 | scaffold_3 | 115750196 | 115750260 |
| scaffold_2 | 50421541 | 50421665 | scaffold_1 | 147219793 | 147220137 |
| scaffold_22 | 29532421 | 29532895 | scaffold_19 | 2338298 | 2338596 |
| scaffold_38 | 2269741 | 2270047 | scaffold_1 | 161713879 | 161713984 |
| scaffold_21 | 29866209 | 29866421 | scaffold_39 | 4602534 | 4602598 |
| scaffold_54 | 2515721 | 2515764 | scaffold_14 | 11750971 | 11751304 |
| scaffold_0 | 25949303 | 25949594 | scaffold_6 | 66885025 | 66885493 |
| scaffold_35 | 13205057 | 13205465 | scaffold_2 | 8247361 | 8247759 |
| scaffold_37 | 12066215 | 12066671 | scaffold_0 | 177213470 | 177213608 |
| scaffold_22 | 12939539 | 12939845 | scaffold_39 | 5248125 | 5248496 |
| scaffold_44 | 8069279 | 8069312 | scaffold_9 | 24985783 | 24986074 |
| scaffold_6 | 60778300 | 60778324 | scaffold_6 | 72646272 | 72646537 |
| scaffold_19 | 18239123 | 18239497 | scaffold_6 | 16347716 | 16347802 |
| scaffold_0 | 18663494 | 18663945 | scaffold_110 | 113119 | 113421 |
| scaffold_10 | 11412124 | 11412475 | scaffold_27 | 5171198 | 5171687 |
| scaffold_9 | 71462 | 71763 | scaffold_9 | 6180957 | 6181129 |
| scaffold_1 | 112831481 | 112831557 | scaffold_10 | 14215150 | 14215392 |
| scaffold_26 | 7687221 | 7687397 | scaffold_20 | 21187571 | 21187927 |
| scaffold_7 | 65770953 | 65771379 | scaffold_19 | 34385730 | 34385826 |
| scaffold_3 | 118117169 | 118117489 | scaffold_3 | 97436277 | 97436398 |
| scaffold_2 | 6797731 | 6797826 | scaffold_35 | 13446725 | 13446964 |
| scaffold_0 | 134717628 | 134718015 | scaffold_36 | 15105267 | 15105429 |
| scaffold_12 | 53824934 | 53825407 | scaffold_9 | 9490960 | 9491438 |
| scaffold_1 | 37231683 | 37231892 | scaffold_0 | 10930489 | 10930570 |
| scaffold_4 | 36243642 | 36243805 | scaffold_9 | 29181906 | 29182190 |
| scaffold_7 | 62301087 | 62301555 | scaffold_3 | 20007124 | 20007431 |
| scaffold_29 | 22627871 | 22628150 | scaffold_12 | 48007028 | 48007336 |
| scaffold_1 | 149299846 | 149300033 | scaffold_51 | 745700 | 746028 |
| scaffold_67 | 1955703 | 1955843 | scaffold_12 | 52908876 | 52909134 |
| scaffold_1 | 130672847 | 130673013 | scaffold_57 | 1909218 | 1909382 |
| scaffold_1 | 40142460 | 40142639 | scaffold_0 | 211532586 | 211532636 |
| scaffold_3 | 63860745 | 63861232 | scaffold_15 | 12787266 | 12787682 |
| scaffold_21 | 10588291 | 10588338 | scaffold_22 | 12856162 | 12856490 |
| scaffold_12 | 36695387 | 36695839 | scaffold_5 | 56486953 | 56487265 |
| scaffold_3 | 78726601 | 78726909 | scaffold_8 | 17571055 | 17571111 |
| scaffold_7 | 38240253 | 38240638 | scaffold_8 | 38858091 | 38858336 |
| chr | start | end | chr | start | end |
| scaffold_30 | 19817051 | 19817193 | scaffold_29 | 14052293 | 14052622 |
| scaffold_44 | 375690 | 375913 | scaffold_21 | 29646970 | 29647160 |
| scaffold_24 | 1318846 | 1319229 | scaffold_8 | 1476104 | 1476312 |
| scaffold_7 | 66072324 | 66072517 | scaffold_14 | 32523357 | 32523446 |
| scaffold_0 | 221591372 | 221591408 | scaffold_20 | 11199461 | 11199739 |
| scaffold_10 | 58195034 | 58195135 | scaffold_345 | 36967 | 37105 |
| scaffold_8 | 71897332 | 71897563 | scaffold_30 | 2168022 | 2168399 |
| scaffold_0 | 161268656 | 161268801 | scaffold_43 | 8957333 | 8957525 |
| scaffold_0 | 46663770 | 46663909 | scaffold_2 | 2765821 | 2765961 |
| scaffold_4 | 5608305 | 5608411 | scaffold_5 | 24051022 | 24051499 |
| scaffold_29 | 23120677 | 23120919 | scaffold_8 | 7746474 | 7746733 |
| scaffold_42 | 7419267 | 7419350 | scaffold_36 | 8311841 | 8312182 |
| scaffold_6 | 50789763 | 50789821 | scaffold_65 | 639323 | 639440 |
| scaffold_1 | 40394347 | 40394539 | scaffold_6 | 63673676 | 63673987 |
| scaffold_4 | 4160072 | 4160309 | scaffold_9 | 35100115 | 35100265 |
| scaffold_19 | 20192868 | 20193186 | scaffold_14 | 39602636 | 39602778 |
| scaffold_6 | 63726426 | 63726528 | scaffold_9 | 3258551 | 3258794 |
| scaffold_5 | 5246222 | 5246424 | scaffold_8 | 17469469 | 17469598 |
| scaffold_13 | 33985232 | 33985382 | scaffold_8 | 20423813 | 20424136 |
| scaffold_20 | 10454055 | 10454187 | scaffold_16 | 34346826 | 34346910 |
| scaffold_0 | 36079659 | 36079667 | scaffold_13 | 1641904 | 1642031 |
| scaffold_0 | 26801285 | 26801377 | scaffold_51 | 3948761 | 3949034 |
| scaffold_34 | 14457203 | 14457559 | scaffold_37 | 13996342 | 13996516 |
| scaffold_30 | 1812957 | 1812983 | scaffold_5 | 9942046 | 9942151 |
| scaffold_43 | 1484770 | 1485027 | scaffold_30 | 11410077 | 11410340 |
| scaffold_1 | 132260978 | 132261193 | scaffold_64 | 217088 | 217521 |
| scaffold_12 | 54208084 | 54208161 | scaffold_16 | 37899062 | 37899144 |
| scaffold_6 | 58076177 | 58076403 | scaffold_42 | 4398746 | 4398817 |
| scaffold_7 | 17882389 | 17882563 | scaffold_8 | 65830515 | 65830720 |
| scaffold_35 | 14679336 | 14679438 | scaffold_10 | 763216 | 763322 |
| scaffold_33 | 7310947 | 7311068 | scaffold_14 | 20648701 | 20648869 |
| scaffold_22 | 32895517 | 32895962 | scaffold_9 | 42494900 | 42495070 |
| scaffold_2 | 46646070 | 46646163 | scaffold_0 | 202845295 | 202845368 |
| scaffold_9 | 53765494 | 53765684 | scaffold_33 | 15811509 | 15811636 |
| scaffold_26 | 7603316 | 7603392 | scaffold_20 | 20189316 | 20189490 |
| scaffold_15 | 24566050 | 24566235 | scaffold_0 | 137115000 | 137115169 |

TABLE 2b
List of differentially methylated regions
(DMRs) identified in IGF-1 adapted samples.

| chr | start | end | chr | start | end |
|---|---|---|---|---|---|
| scaffold_4 | 46838477 | 46838525 | scaffold_0 | 127708478 | 127708500 |
| scaffold_1 | 40452365 | 40452454 | scaffold_12 | 28190332 | 28190769 |
| scaffold_4 | 31231044 | 31231111 | scaffold_4 | 39792499 | 39792654 |
| scaffold_59 | 2358940 | 2359226 | scaffold_5 | 3862876 | 3863105 |
| scaffold_2 | 40270729 | 40271062 | scaffold_4 | 82124551 | 82124691 |
| scaffold_21 | 33819495 | 33819720 | scaffold_15 | 10894110 | 10894291 |
| scaffold_36 | 554210 | 554243 | scaffold_67 | 566792 | 566824 |
| scaffold_0 | 24828369 | 24828530 | scaffold_2 | 13520901 | 13521043 |
| scaffold_0 | 84626749 | 84626904 | scaffold_4 | 38044709 | 38044879 |
| scaffold_15 | 23110977 | 23111032 | scaffold_24 | 26202059 | 26202329 |
| scaffold_51 | 1029523 | 1029755 | scaffold_27 | 875976 | 876003 |
| scaffold_54 | 2943156 | 2943282 | scaffold_45 | 2128856 | 2128976 |
| scaffold_30 | 12227834 | 12227959 | scaffold_37 | 2439671 | 2439833 |
| scaffold_20 | 5206974 | 5207142 | scaffold_21 | 21559867 | 21559952 |
| scaffold_8 | 27498735 | 27498750 | scaffold_8 | 65007115 | 65007319 |
| scaffold_6 | 23334205 | 23334252 | scaffold_8 | 53964131 | 53964301 |
| scaffold_21 | 115674 | 115770 | scaffold_3 | 58320469 | 58320666 |
| scaffold_5 | 71484400 | 71484510 | scaffold_16 | 20587686 | 20587832 |
| scaffold_5 | 76487736 | 76487905 | scaffold_43 | 5979577 | 5979789 |
| scaffold_294 | 30527 | 30664 | scaffold_14 | 35705378 | 35705569 |
| scaffold_18 | 362609 | 362731 | scaffold_17 | 9641455 | 9641523 |
| scaffold_39 | 5115921 | 5115984 | scaffold_6015 | 205 | 285 |
| scaffold_0 | 145623209 | 145623306 | scaffold_2 | 4637629 | 4637789 |
| scaffold_17 | 37526021 | 37526026 | scaffold_9 | 15607400 | 15607517 |
| scaffold_5 | 62406675 | 62406812 | scaffold_12 | 47812083 | 47812228 |
| scaffold_10 | 40092253 | 40092370 | scaffold_3 | 24837697 | 24837987 |
| scaffold_12 | 34496664 | 34496798 | scaffold_8 | 67097612 | 67097829 |
| scaffold_17 | 37823878 | 37823900 | scaffold_7 | 12194341 | 12194440 |
| scaffold_177 | 108918 | 109057 | scaffold_27 | 3759367 | 3759442 |
| scaffold_43 | 2789548 | 2789652 | scaffold_30 | 6458299 | 6458317 |
| scaffold_5891 | 625 | 1001 | scaffold_4 | 17114849 | 17114949 |
| scaffold_1 | 15992497 | 15992536 | scaffold_24 | 15714804 | 15714866 |
| scaffold_12 | 19536793 | 19536897 | scaffold_16 | 39261687 | 39261846 |
| scaffold_44 | 2759370 | 2759457 | scaffold_29 | 14743908 | 14743981 |
| scaffold_0 | 208515585 | 208515621 | scaffold_19 | 5923042 | 5923155 |
| scaffold_0 | 44169368 | 44169524 | scaffold_24 | 22880118 | 22880129 |
| chr | start | end | chr | start | end |
| scaffold_10 | 1691212 | 1691335 | scaffold_19 | 6140711 | 6140830 |
| scaffold_5 | 24215603 | 24215652 | scaffold_0 | 50668434 | 50668441 |
| scaffold_3 | 1539960 | 1539970 | scaffold_31 | 19178925 | 19179027 |
| scaffold_22 | 7204758 | 7204837 | scaffold_33 | 960482 | 960554 |
| scaffold_6 | 69128866 | 69129097 | scaffold_0 | 8374159 | 8374248 |
| scaffold_5 | 56754022 | 56754033 | scaffold_24 | 10598314 | 10598477 |
| scaffold_2 | 25801677 | 25801855 | scaffold_0 | 9596052 | 9596059 |
| scaffold_2760 | 2736 | 2905 | scaffold_6970 | 2067 | 2165 |
| scaffold_30 | 9841394 | 9841503 | scaffold_39 | 6392428 | 6392478 |
| scaffold_13 | 14205334 | 14205434 | scaffold_16 | 35401569 | 35401615 |
| scaffold_62 | 366037 | 366198 | scaffold_2 | 46436059 | 46436218 |
| scaffold_9 | 63865309 | 63865381 | scaffold_7 | 34196115 | 34196250 |
| scaffold_21 | 33052401 | 33052490 | scaffold_2 | 96740755 | 96740864 |
| scaffold_12 | 18894036 | 18894186 | scaffold_20 | 21182361 | 21182517 |
| scaffold_0 | 4927850 | 4927991 | scaffold_0 | 129535903 | 129536004 |
| scaffold_1 | 112272095 | 112272230 | scaffold_7 | 46637297 | 46637376 |
| scaffold_16 | 11622901 | 11623001 | scaffold_4 | 38333552 | 38333613 |
| scaffold_12 | 24914255 | 24914481 | scaffold_6 | 64720336 | 64720469 |
| scaffold_2 | 3617470 | 3617546 | scaffold_0 | 188369396 | 188369480 |
| scaffold_0 | 128210855 | 128211154 | scaffold_1 | 65565559 | 65565649 |
| scaffold_27 | 16149908 | 16150010 | scaffold_9 | 14408679 | 14408926 |
| scaffold_0 | 207596754 | 207596781 | scaffold_26 | 7686779 | 7686807 |
| scaffold_12 | 42555855 | 42556032 | scaffold_20 | 24856220 | 24856353 |
| scaffold_0 | 36074997 | 36075058 | scaffold_47 | 2725859 | 2726019 |
| scaffold_16 | 20587336 | 20587379 | scaffold_133 | 66237 | 66309 |
| scaffold_41 | 8837831 | 8837936 | scaffold_6 | 32500581 | 32500670 |
| scaffold_21 | 32448146 | 32448250 | scaffold_3 | 35301312 | 35301413 |
| scaffold_2589 | 1120 | 1221 | scaffold_5 | 594905 | 595006 |
| scaffold_1 | 53918654 | 53918783 | scaffold_21 | 30139718 | 30139777 |
| scaffold_5 | 77039891 | 77039983 | scaffold_3 | 37045604 | 37045685 |
| scaffold_5 | 57947700 | 57947908 | scaffold_7 | 68766393 | 68766459 |
| scaffold_3 | 73955480 | 73955543 | scaffold_5 | 6745694 | 6745839 |
| scaffold_20 | 29518333 | 29518425 | scaffold_1 | 32178748 | 32178866 |
| scaffold_777 | 4190 | 4244 | scaffold_0 | 138909478 | 138909547 |
| scaffold_29 | 23616788 | 23616848 | scaffold_8 | 39479084 | 39479159 |
| scaffold_46 | 1615662 | 1615869 | scaffold_0 | 27967626 | 27967732 |
| | | | scaffold_35 | 11680925 | 11680962 |

### Example 3

Detection of Heterologous Protein Quality from CHO Cells

### Wet-Lab Methodology

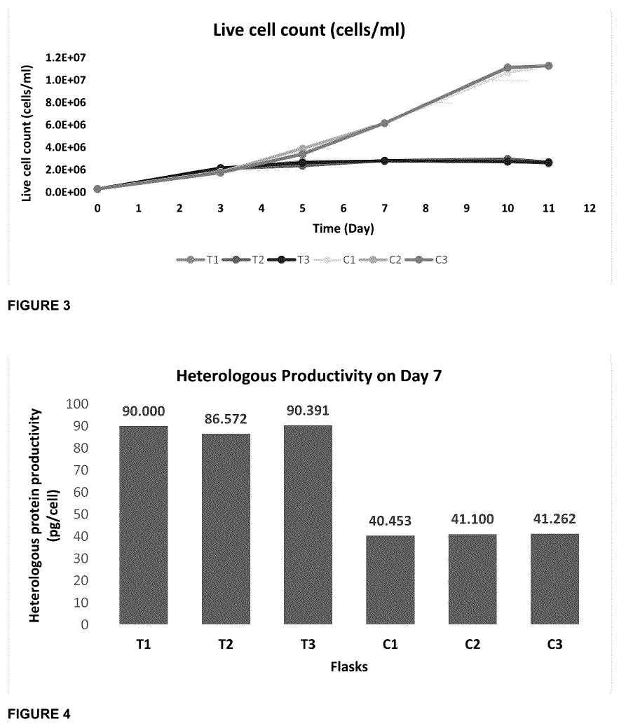

For this experiment, a transgenic CHO cell line, Humira431 clone (acquired from A*Star BTI), was grown in EX-Cell Advanced Fed-batch medium supplemented with 6 mM L-glutamine at 37° C., 8% CO2, at a shaking speed of 150 RPM. Fed-Batch culture of 6 flasks was maintained for 11 days where 3 flasks represent technical replicates for the control set (C1, C2, C3) and 3 flasks represent technical replicates for the treatment set (T1, T2, T3). The flasks were seeded with 3E5 viable cells/mL on day 0 and the culture was fed with EX-CELL® Advanced CHO Feed 1 on day 3, 5, 7, 9 and glucose was topped up to 6 g/l using 45% glucose when dropped below 3 g/l. To induce hyperosmolarity in the cell culture media, concentrated Sodium chloride solution was added on day 3 to the treatment set leading to an increase of the osmolarity of the media from 320 mOsm/kg to 480 mOsm/kg. Cell count, cell viability, and heterologous protein production were measured every 2 days and cell pellets were collected for both control and treatment set on day 7. Induction of hyperosmolarity in CHO cell media by Sodium Chloride resulted in reduced growth rate (FIG. 3), increase in heterologous protein productivity (FIG. 4) and alteration in the relative abundance of each N-glycans modifications (Table 3) for treatment set as compared to the control set. Alternation in the relative abundance of each N-glycans symbolizes a change in the heterologous protein quality.

### DNA Extraction

DNA is extracted using the PureLink Genomic DNA Isolation Minikit kit (Invitrogen), including RNAase treatment following the manufacturer's instructions. DNA quantity is measured by PicoGreen assay and DNA quality is assessed via NanoDrop (Thermo Scientific) to ensure the A260/280 ratio is ≤1.8. A small amount of sample is then also analysed using automated electrophoresis on TapeStation (Agilent) to ensure each sample contains high molecular weight DNA.

### Sequencing Analysis

The genomic DNA (500 ng) from the samples were used to prepare libraries for Whole Genome Bisulfite Sequencing (WGBS). The sequencing of the libraries was performed by a third party on a NovaSeq platform which generated 125 GB data per sample with 20× coverage.

### Data Processing:

### Processing & Analysis of Sequencing Data:

Raw sequencing data were conducted quality control (fastqc) 1, sequencing adaptors trimming (TrimGalore)2, and alignment with Bismark3.

Bismark was also used for removing duplicated reads and extracting methylation counts from alignment output. SNPs were filtered out, and only counts with a minimum coverage of 10× were used for the downstream analysis.

The methylation ratio of the Control (C1) and Treatment (T1) samples were then extracted. The sites with a methylation difference of 30% were then filtered. (Table 4)

These methylation sites may be indicative of difference in protein quality between the samples.

TABLE 3
Comparison of percentage of N-Glycan modifications
abundance between control (C1) and Treated (T1)

| | | | Relative | Relative |
|---|---|---|---|---|
| | | Relevant genes | abundance | abundance |
| N-Glycans | | responsible for | in control | in treated |
| modification | Role | modification | (C1) | (T1) |
| Fucosylation | Affects antibody- | Fut8 | 97.19 | 93.58 |
| | dependent cell- | | | |
| | mediated cytotoxicity | | | |
| Galactosylation | Affects complement- | B4galt1, B4galt2, | 50.05 | 44.63 |
| | dependent cytotoxicity | B4galt3, B4galt4, | | |
| | | B4galt5, Gale | | |
| High mannose | Affects antibody | Man1a1, Man1b, | 2.81 | 6.42 |
| | clearance and therefore | Man1c1, Man2a1, | | |
| | antibody efficacy | Man2a2, Man2b1, | | |
| | | Man2c1, Manbal, | | |
| | | Manea, Mgat4d | | |
| Sialylation | Affects antibody half- | Nans, Nanp, Slc35a1, | 2.44 | 1.39 |
| | life and therefore | St3gal4, St3gal5, | | |
| | antibody efficacy | St3gal6, St6gal2, | | |
| | | St3gal1, St3gal2, | | |
| | | Cmas, Gne | | |

TABLE 4a
CpG sites of the genes from Table 3 that with
a methylation difference of 30% and more.

| Chrom | Position | gene name |
|---|---|---|
| NW_023276806.1 | 194034622 | SLC35B4 |
| NW_023276806.1 | 194037022 | SLC35B4 |
| NW_023276806.1 | 194040212 | SLC35B4 |
| NW_023276807.1 | 4438290 | ST6GAL2 |
| NW_023276807.1 | 4440888 | ST6GAL2 |
| NW_023276807.1 | 4445039 | ST6GAL2 |
| NW_023276807.1 | 4445063 | ST6GAL2 |
| NW_023276807.1 | 4461289 | ST6GAL2 |
| NW_023276807.1 | 4465462 | ST6GAL2 |
| NW_023276807.1 | 4476321 | ST6GAL2 |
| NW_023276807.1 | 4462633 | ST6GAL2 |
| NW_023276807.1 | 10707927 | MGAT4A |
| NW_023276807.1 | 10733025 | MGAT4A |
| NW_023276807.1 | 10733043 | MGAT4A |
| NW_023276807.1 | 10735731 | MGAT4A |
| NW_023276807.1 | 10741259 | MGAT4A |
| NW_023276807.1 | 10752780 | MGAT4A |
| NW_023276807.1 | 10769338 | MGAT4A |
| NW_023276807.1 | 83989704 | B3GNT2 |

TABLE 4b
CpG sites of the genes from Table 3 that with
a methylation difference of 30% and more.

| Chrom | Position | gene name | Chrom | Position | gene name |
|---|---|---|---|---|---|
| NC_048595.1 | 99690229 | GNE | NC_048595.1 | 18583642 | MAN1C1 |
| NC_048595.1 | 99707995 | GNE | NC_048595.1 | 18589149 | MAN1C1 |
| NC_048595.1 | 99715144 | GNE | NC_048595.1 | 18598510 | MAN1C1 |
| NC_048595.1 | 99719272 | GNE | NC_048595.1 | 18609419 | MAN1C1 |
| NC_048595.1 | 101761968 | B4GALT1 | NC_048595.1 | 18609431 | MAN1C1 |
| NC_048595.1 | 101784566 | B4GALT1 | NC_048595.1 | 18636073 | MAN1C1 |
| NC_048595.1 | 101786273 | B4GALT1 | NC_048595.1 | 18647950 | MAN1C1 |
| NC_048595.1 | 107356303 | SLC35A1 | NC_048595.1 | 18662447 | MAN1C1 |
| NC_048595.1 | 107359073 | SLC35A1 | NC_048595.1 | 18664101 | MAN1C1 |
| NC_048595.1 | 107369613 | SLC35A1 | NC_048595.1 | 18669934 | MAN1C1 |
| NC_048595.1 | 254993626 | MAN1A1 | NC_048595.1 | 18673975 | MAN1C1 |
| NC_048595.1 | 254993626 | MAN1A | NC_048595.1 | 18677085 | MAN1C1 |
| NC_048595.1 | 254993726 | MAN1A1 | NC_048595.1 | 18678337 | MAN1C1 |
| NC_048595.1 | 254993726 | MAN1A | NC_048595.1 | 18697060 | MAN1C1 |
| NC_048595.1 | 254995034 | MAN1A1 | NC_048595.1 | 18699984 | MAN1C1 |
| NC_048595.1 | 254995034 | MAN1A | NC_048595.1 | 18704477 | MAN1C1 |
| NC_048595.1 | 255034337 | MAN1A1 | NC_048595.1 | 18704723 | MAN1C1 |
| NC_048595.1 | 255034337 | MAN1A | NC_048595.1 | 18715037 | MAN1C1 |
| NC_048595.1 | 255142336 | MAN1A1 | NC_048595.1 | 18715096 | MAN1C1 |
| NC_048595.1 | 255142336 | MAN1A | NC_048595.1 | 28196001 | MANEA |
| NC_048595.1 | 453695480 | MAN2A1 | NC_048595.1 | 34447035 | B4GALT2 |
| NC_048595.1 | 453719166 | MAN2A1 | NC_048595.1 | 96929050 | NANS |
| NC_048595.1 | 453743262 | MAN2A1 | NC_048595.1 | 99454025 | GNE |
| NC_048595.1 | 453761810 | MAN2A1 | NC_048595.1 | 99457674 | GNE |
| NC_048595.1 | 453799399 | MAN2A1 | NC_048595.1 | 99468137 | GNE |
| NC_048595.1 | 453805446 | MAN2A1 | NC_048595.1 | 99497688 | GNE |
| NC_048595.1 | 453819903 | MAN2A1 | NC_048595.1 | 99505654 | GNE |
| NC_048595.1 | 453826615 | MAN2A1 | NC_048595.1 | 99512073 | GNE |
| NC_048595.1 | 453831470 | MAN2A1 | NC_048595.1 | 99548650 | GNE |
| NC_048595.1 | 453843302 | MAN2A1 | NC_048595.1 | 99525120 | GNE |
| NC_048596.1 | 117240361 | MAN2A2 | NC_048595.1 | 99538592 | GNE |
| NC_048596.1 | 158998087 | MGAT4D | NC_048595.1 | 99565150 | GNE |
| NC_048596.1 | 159005743 | MGAT4D | NC_048595.1 | 99592802 | GNE |
| NC_048596.1 | 159273924 | MAN2B1 | NC_048595.1 | 99594725 | GNE |
| NC_048596.1 | 187370581 | ST3GAL2 | NC_048595.1 | 99595184 | GNE |
| NC_048596.1 | 274579270 | B4GALT7 | NC_048595.1 | 99595226 | GNE |
| NC_048596.1 | 274592442 | B4GALT7 | NC_048595.1 | 99602940 | GNE |
| NC_048596.1 | 274593274 | B4GALT7 | NC_048595.1 | 99616272 | GNE |
| NC_048596.1 | 274618915 | B4GALT7 | NC_048595.1 | 99617813 | GNE |
| NC_048596.1 | 274622333 | B4GALT7 | NC_048595.1 | 99618422 | GNE |
| NC_048596.1 | 274622855 | B4GALT7 | NC_048595.1 | 99618659 | GNE |
| NC_048596.1 | 274632778 | B4GALT7 | NC_048595.1 | 99626534 | GNE |
| NC_048596.1 | 274632779 | B4GALT7 | NC_048595.1 | 99641777 | GNE |

TABLE 4c
CpG sites of the genes from Table 3 that with
a methylation difference of 30% and more.

| Chrom | Position | gene name | Chrom | Position | gene name |
|---|---|---|---|---|---|
| NC_048596.1 | 274636389 | B4GALT7 | NC_048599.1 | 58084545 | GANC |
| NC_048596.1 | 274636517 | B4GALT7 | NC_048599.1 | 58088435 | GANC |
| NC_048597.1 | 33843653 | ST3GAL6 | NC_048599.1 | 136689191 | MAN1B |
| NC_048597.1 | 33844529 | ST3GAL6 | NC_048599.1 | 136692935 | MAN1B |
| NC_048597.1 | 33846332 | ST3GAL6 | NC_048600.1 | 129489374 | MGAT5B |
| NC_048597.1 | 33873495 | ST3GAL6 | NC_048600.1 | 129501536 | MGAT5B |
| NC_048597.1 | 33895658 | ST3GAL6 | NC_048600.1 | 129505548 | MGAT5B |
| NC_048597.1 | 147439772 | ST3GAL4 | NC_048600.1 | 129521011 | MGAT5B |
| NC_048597.1 | 168666631 | MAN2C1 | NC_048600.1 | 129537369 | MGAT5B |
| NC_048597.1 | 168667528 | MAN2C1 | NC_048601.1 | 6824946 | CMAS |
| NC_048597.1 | 168671477 | MAN2C1 | NC_048601.1 | 63832889 | ST3GAL5 |
| NC_048597.1 | 208024327 | A4GNT | NC_048601.1 | 63857937 | ST3GAL5 |
| NC_048598.1 | 61327964 | MGAT5 | NW_023276806.1 | 41521292 | SLC35A3 |
| NC_048598.1 | 61336591 | MGAT5 | NW_023276806.1 | 41548633 | SLC35A3 |
| NC_048598.1 | 61337163 | MGAT5 | NW_023276806.1 | 41552111 | SLC35A3 |
| NC_048598.1 | 61351895 | MGAT5 | NW_023276806.1 | 41557468 | SLC35A3 |
| NC_048598.1 | 61358384 | MGAT5 | NW_023276806.1 | 167654763 | MGAT4C |
| NC_048598.1 | 61394081 | MGAT5 | NW_023276806.1 | 167654763 | MGAT4C |
| NC_048598.1 | 61429100 | MGAT5 | NW_023276806.1 | 193807458 | SLC35B4 |
| NC_048598.1 | 61451063 | MGAT5 | NW_023276806.1 | 193824654 | SLC35B4 |
| NC_048598.1 | 61492346 | MGAT5 | NW_023276806.1 | 193832882 | SLC35B4 |
| NC_048598.1 | 61542077 | MGAT5 | NW_023276806.1 | 193841067 | SLC35B4 |
| NC_048598.1 | 61543813 | MGAT5 | NW_023276806.1 | 193856535 | SLC35B4 |
| NC_048598.1 | 61563732 | MGAT5 | NW_023276806.1 | 193858400 | SLC35B4 |
| NC_048598.1 | 61586227 | MGAT5 | NW_023276806.1 | 193862096 | SLC35B4 |
| NC_048598.1 | 61593379 | MGAT5 | NW_023276806.1 | 193865176 | SLC35B4 |
| NC_048598.1 | 61594613 | MGAT5 | NW_023276806.1 | 193866729 | SLC35B4 |
| NC_048598.1 | 126107667 | FUT8 | NW_023276806.1 | 193888769 | SLC35B4 |
| NC_048598.1 | 126188377 | FUT8 | NW_023276806.1 | 193889641 | SLC35B4 |
| NC_048598.1 | 126269216 | FUT8 | NW_023276806.1 | 193895366 | SLC35B4 |
| NC_048598.1 | 126269245 | FUT8 | NW_023276806.1 | 193896660 | SLC35B4 |
| NC_048599.1 | 12094759 | B4GALT5 | NW_023276806.1 | 193903403 | SLC35B4 |
| NC_048599.1 | 22163147 | MANBAL | NW_023276806.1 | 193916984 | SLC35B4 |
| NC_048599.1 | 58030897 | GANC | NW_023276806.1 | 193924435 | SLC35B4 |
| NC_048599.1 | 58033672 | GANC | NW_023276806.1 | 193938426 | SLC35B4 |
| NC_048599.1 | 58037340 | GANC | NW_023276806.1 | 193944484 | SLC35B4 |
| NC_048599.1 | 58042276 | GANC | NW_023276806.1 | 193953141 | SLC35B4 |
| NC_048599.1 | 58048007 | GANC | NW_023276806.1 | 193954030 | SLC35B4 |
| NC_048599.1 | 58048159 | GANC | NW_023276806.1 | 193950259 | SLC35B4 |
| NC_048599.1 | 58049932 | GANC | NW_023276806.1 | 193962623 | SLC35B4 |
| NC_048599.1 | 58057978 | GANC | NW_023276806.1 | 193990972 | SLC35B4 |
| NC_048599.1 | 58067312 | GANC | NW_023276806.1 | 193995911 | SLC35B4 |
| NC_048599.1 | 58076632 | GANC | NW_023276806.1 | 194020307 | SLC35B4 |

### Example 4

Detection of Heterologous Protein Quantity from CHO Cells

### Wet-Lab Methodology

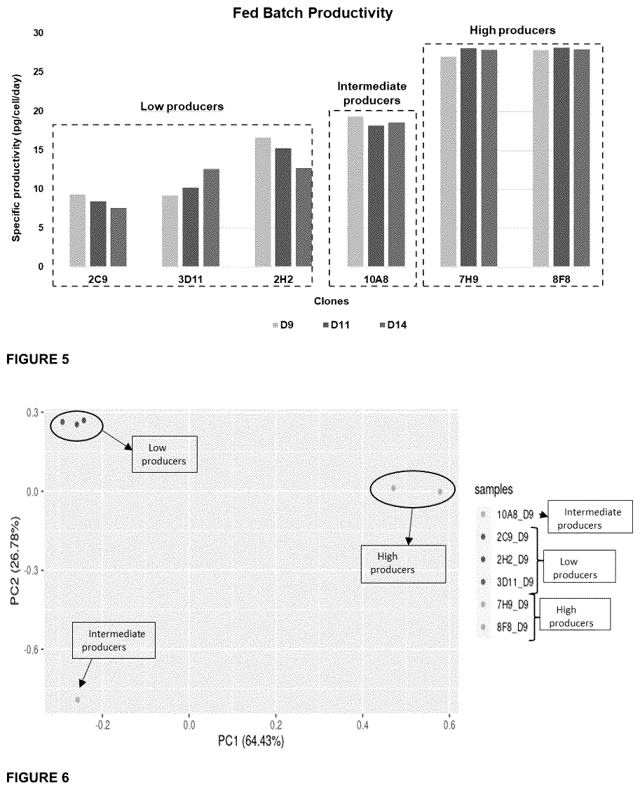

For this experiment, five transgenic CHO clones (acquired from A*Star BTI) were grown in EX-Cell Advanced Fed-batch medium supplemented with 6 mM L-glutamine at 37° C., 8% CO2, at a shaking speed of 225 RPM. The five transgenic CHO cell lines include low producers (3D11, 2C9, 2H2), intermediate producer (10A8) and high producers (8F8, 7H9). The flasks were seeded with 3E5 viable cells/mL on day 0 and the culture was fed with Cell Boost 7a on Day 3, 5, 7, 9, 11 and glucose was topped up to 6 g/l using 45% glucose when dropped below 2 g/l. The fed-Batch culture of 6 clones was maintained for 14 days. Cell count, cell viability, and heterologous protein production were measured every 2 days and cell pellets were collected on day 9. Specific productivity (pg/cell/day) for all the 6 clones was calculated for day 9, 11 and 14 as shown in FIG. 5.

### DNA Extraction

DNA is extracted using the PureLink Genomic DNA Isolation Minikit kit (Invitrogen), including RNAase treatment following the manufacturer's instructions. DNA quantity is measured by PicoGreen assay and DNA quality is assessed via NanoDrop (Thermo Scientific) to ensure the A260/280 ratio is ≤1.8. A small amount of sample is then also analysed using automated electrophoresis on TapeStation (Agilent) to ensure each sample contains high molecular weight DNA.

### Bisulfite Conversion and BeadChip Analysis

The genomic DNA samples are then subjected to bisulfite conversion using the EZ DNA Methylation-Gold™ Kit (Zymo Research). The methylation levels are then quantified using our customized methylation BeadChip kits (Illumina) which can analyze over 50,000 methylation sites quantitatively across the genome at single-nucleotide resolution. After bisulfite conversion, samples were processed through a three-day workflow including sample amplification, fragmentation, precipitation, hybridization to BeadChip and X-stain according to Infinium HD Methylation Assay (Illumina, Document #15019519 v07), before being imaged on the iScan (Illumina) where intensity files for the computation of beta values are generated.

### Data Processing:

### Processing of Beadchip Data:

The customized chip array data processing is performed in R version 4.1.2 using sesame version 1.14.2. DNA methylation level for each site was calculated as methylation B-value. Beta values are defined as methylated signal/(methylated signal+unmethylated signal). It can be computed using getBetas function. The SeSAMe pipeline (Zhou et al. 2018) was used to generate normalized B-values and for quality control. Low intensity-based detection calling and making (based on p-value) was done with pOOBAH. Background subtraction based on normal-exponential deconvolution using out-of-band probes noob (Triche et al. 2013) and optionally with extra bleed-through subtraction were also implemented.

After obtaining the beta values, control probes were filtered out of the data frame. CpG sites with NA beta values were also removed from the data frame

To obtain Differentially Methylated Positions (DMPs) between high protein productivity clones (7H9 & 8F8) and low protein productivity clones (2C9, 2H2 & 3D11), sample 10A8 was excluded from the beta value data frame prior to extracting the DMPs. After filtering out 10A8, DMPs between high protein productivity clones and low protein productivity clones were extracted using the dml and dmr function from the sesame package. The dmr function will result in a data frame and to obtain the more statistically significant DMPs, only DMPs with Pr(|t|)<0.05 were retained while the rest were removed from the data frame. This resulted in 901 CpG sites (after removing probes with NA) remaining and the PCA plot for these sites were plotted using the prcomp followed by autoplot functions. These cites are shown in Table 5.

TABLE 5a
901 CpG sites from CHO cells relevant for the method
according to any aspect of the present invention.

| Chrom | Position |
|---|---|
| chrM | 7066 |
| NW_003613580v1 | 3333804 |
| NW_003613580v1 | 3954428 |
| NW_003613581v1 | 789418 |
| NW_003613581v1 | 789442 |
| NW_003613581v1 | 2129902 |
| NW_003613581v1 | 3347656 |
| NW_003613583v1 | 742781 |
| NW_003613583v1 | 4072955 |
| NW_003613584v1 | 1804208 |
| NW_003613584v1 | 4874590 |
| NW_003613584v1 | 4968470 |
| NW_003613585v1 | 1712455 |
| NW_003613585v1 | 4588331 |
| NW_003613587v1 | 1803863 |
| NW_003613588v1 | 4150258 |
| NW_003613591v1 | 443072 |
| NW_003613591v1 | 443389 |
| NW_003613591v1 | 4480091 |
| NW_003613593v1 | 2514807 |
| NW_003613594v1 | 2165009 |
| NW_003613595v1 | 1891793 |
| NW_003613595v1 | 2628153 |
| NW_003613595v1 | 4112020 |
| NW_003613595v1 | 4275041 |
| NW_003613598v1 | 340147 |
| NW_003613598v1 | 471687 |
| NW_003613598v1 | 1035832 |
| NW_003613598v1 | 1165984 |
| NW_003613598v1 | 2068411 |
| NW_003613598v1 | 2420965 |
| NW_003613598v1 | 2420979 |
| NW_003613598v1 | 2420986 |
| NW_003613599v1 | 676136 |
| NW_003613599v1 | 1348737 |
| NW_003613599v1 | 4572911 |
| NW_003613600v1 | 3978802 |
| NW_003613601v1 | 123462 |
| NW_003613601v1 | 4411385 |
| NW_003613601v1 | 4531976 |
| NW_003613602v1 | 3554981 |
| NW_003613605v1 | 207494 |
| NW_003613605v1 | 207497 |
| NW_003613605v1 | 235049 |
| NW_003613605v1 | 2991156 |
| NW_003613605v1 | 4499253 |
| NW_003613605v1 | 4499464 |
| NW_003613605v1 | 4510789 |
| NW_003613608v1 | 2694669 |
| NW_003613608v1 | 3366418 |
| NW_003613610v1 | 1911108 |
| NW_003613610v1 | 3571261 |
| NW_003613610v1 | 3879511 |
| NW_003613610v1 | 3943585 |
| NW_003613613v1 | 1888797 |
| NW_003613613v1 | 3063777 |
| NW_003613613v1 | 3075341 |
| NW_003613615v1 | 2319896 |
| NW_003613617v1 | 1337762 |
| NW_003613618v1 | 56689 |
| NW_003613618v1 | 382594 |
| NW_003613618v1 | 938265 |
| NW_003613618v1 | 2966410 |
| NW_003613619v1 | 1456382 |
| NW_003613619v1 | 1456520 |
| NW_003613619v1 | 1873501 |
| NW_003613619v1 | 2077678 |
| NW_003613620v1 | 1426835 |
| NW_003613621v1 | 658138 |
| NW_003613621v1 | 1348067 |
| NW_003613622v1 | 704511 |
| NW_003613622v1 | 3499751 |
| NW_003613624v1 | 3290771 |
| NW_003613627v1 | 3085809 |
| NW_003613628v1 | 2762665 |
| NW_003613628v1 | 2834300 |
| NW_003613629v1 | 1359917 |
| NW_003613630v1 | 302587 |
| NW_003613630v1 | 342701 |
| NW_003613630v1 | 2058978 |
| NW_003613630v1 | 2598722 |
| NW_003613630v1 | 3111171 |
| NW_003613631v1 | 1656238 |
| NW_003613632v1 | 90703 |
| NW_003613632v1 | 90721 |
| NW_003613632v1 | 3176895 |
| NW_003613633v1 | 118409 |
| NW_003613633v1 | 118686 |
| NW_003613633v1 | 245419 |
| NW_003613633v1 | 2413771 |
| NW_003613633v1 | 2741954 |
| NW_003613635v1 | 2415036 |
| NW_003613635v1 | 3061425 |
| NW_003613637v1 | 387154 |
| NW_003613637v1 | 406413 |
| NW_003613637v1 | 591293 |
| NW_003613637v1 | 778702 |
| NW_003613637v1 | 2190289 |
| NW_003613637v1 | 2528096 |
| NW_003613637v1 | 2737567 |
| NW_003613637v1 | 2820867 |
| NW_003613637v1 | 3445092 |
| NW_003613638v1 | 1429683 |
| NW_003613638v1 | 2956773 |
| NW_003613639v1 | 1805199 |
| NW_003613639v1 | 2975779 |
| NW_003613640v1 | 177694 |
| NW_003613640v1 | 1775049 |
| NW_003613640v1 | 3255106 |
| NW_003613640v1 | 3331386 |
| NW_003613641v1 | 1278865 |
| NW_003613641v1 | 1795685 |
| NW_003613642v1 | 2446263 |
| NW_003613643v1 | 3030300 |
| NW_003613644v1 | 213787 |
| NW_003613646v1 | 18175 |
| NW_003613646v1 | 3026004 |
| NW_003613647v1 | 1739036 |
| NW_003613647v1 | 1739054 |
| NW_003613649v1 | 209622 |
| NW_003613649v1 | 315308 |
| NW_003613650v1 | 2641315 |
| NW_003613652v1 | 1780259 |
| NW_003613655v1 | 480102 |
| NW_003613655v1 | 1026643 |
| NW_003613656v1 | 470840 |
| NW_003613657v1 | 1252778 |
| NW_003613658v1 | 1459637 |
| NW_003613658v1 | 2312007 |
| NW_003613659v1 | 2486251 |
| NW_003613659v1 | 3045055 |
| NW_003613661v1 | 632649 |
| NW_003613664v1 | 1228993 |
| NW_003613664v1 | 1229346 |
| NW_003613664v1 | 1860001 |
| NW_003613665v1 | 35300 |
| NW_003613665v1 | 648271 |
| NW_003613665v1 | 648311 |
| NW_003613665v1 | 951006 |
| NW_003613665v1 | 2005370 |
| NW_003613667v1 | 1909185 |
| NW_003613668v1 | 705672 |
| NW_003613669v1 | 1816220 |
| NW_003613669v1 | 2513015 |
| NW_003613670v1 | 2524510 |
| NW_003613672v1 | 2015155 |
| NW_003613673v1 | 2151213 |
| NW_003613673v1 | 2207443 |
| NW_003613677v1 | 1013980 |
| NW_003613677v1 | 1975645 |
| NW_003613677v1 | 2627367 |
| NW_003613679v1 | 1421655 |
| NW_003613681v1 | 568311 |
| NW_003613681v1 | 1168807 |
| NW_003613681v1 | 1245004 |
| NW_003613681v1 | 1245072 |
| NW_003613681v1 | 1751238 |
| NW_003613681v1 | 1858151 |
| NW_003613681v1 | 2000160 |
| NW_003613682v1 | 2066452 |
| NW_003613682v1 | 2067286 |
| NW_003613683v1 | 1519204 |
| NW_003613684v1 | 841975 |
| NW_003613685v1 | 461887 |
| NW_003613685v1 | 1828629 |
| NW_003613685v1 | 2071193 |
| NW_003613686v1 | 1383834 |
| NW_003613686v1 | 2405978 |
| NW_003613689v1 | 1725989 |
| NW_003613689v1 | 1726878 |
| NW_003613689v1 | 2407714 |
| NW_003613692v1 | 672710 |
| NW_003613692v1 | 711817 |
| NW_003613692v1 | 711826 |
| NW_003613692v1 | 2648451 |
| NW_003613694v1 | 1130815 |

TABLE 5b
901 CpG sites from CHO cells relevant for the method
according to any aspect of the present invention.

| Chrom | Position |
|---|---|
| NW_003613694v1 | 1231754 |
| NW_003613694v1 | 1370567 |
| NW_003613696v1 | 1171629 |
| NW_003613698v1 | 2111797 |
| NW_003613699v1 | 671894 |
| NW_003613699v1 | 773871 |
| NW_003613699v1 | 1208861 |
| NW_003613699v1 | 1257766 |
| NW_003613699v1 | 1506314 |
| NW_003613699v1 | 1358862 |
| NW_003613699v1 | 1753355 |
| NW_003613699v1 | 2246384 |
| NW_003613701v1 | 1717003 |
| NW_003613702v1 | 279393 |
| NW_003613702v1 | 279395 |
| NW_003613702v1 | 1899862 |
| NW_003613704v1 | 1022051 |
| NW_003613704v1 | 1022190 |
| NW_003613705v1 | 600880 |
| NW_003613705v1 | 638694 |
| NW_003613706v1 | 275333 |
| NW_003613706v1 | 952789 |
| NW_003613706v1 | 1880933 |
| NW_003613709v1 | 52344 |
| NW_003613709v1 | 208186 |
| NW_003613709v1 | 684981 |
| NW_003613710v1 | 513381 |
| NW_003613710v1 | 1028244 |
| NW_003613712v1 | 218697 |
| NW_003613716v1 | 222224 |
| NW_003613716v1 | 1058199 |
| NW_003613716v1 | 1058275 |
| NW_003613716v1 | 1219477 |
| NW_003613716v1 | 1219503 |
| NW_003613716v1 | 1237101 |
| NW_003613716v1 | 1843430 |
| NW_003613717v1 | 2415440 |
| NW_003613717v1 | 2415461 |
| NW_003613720v1 | 1231411 |
| NW_003613720v1 | 2334108 |
| NW_003613721v1 | 2363333 |
| NW_003613723v1 | 2208137 |
| NW_003613723v1 | 2217915 |
| NW_003613726v1 | 728442 |
| NW_003613726v1 | 849526 |
| NW_003613726v1 | 967839 |
| NW_003613726v1 | 1473749 |
| NW_003613727v1 | 1301225 |
| NW_003613727v1 | 1301228 |
| NW_003613728v1 | 2203990 |
| NW_003613730v1 | 1653881 |
| NW_003613730v1 | 2087487 |
| NW_003613734v1 | 314611 |
| NW_003613734v1 | 1554784 |
| NW_003613734v1 | 1592600 |
| NW_003613736v1 | 751434 |
| NW_003613737v1 | 1554609 |
| NW_003613737v1 | 2346427 |
| NW_003613738v1 | 355263 |
| NW_003613739v1 | 1976776 |
| NW_003613742v1 | 1733115 |
| NW_003613745v1 | 1605146 |
| NW_003613745v1 | 1755736 |
| NW_003613745v1 | 1755781 |
| NW_003613745v1 | 1831705 |
| NW_003613745v1 | 2105507 |
| NW_003613748v1 | 1109730 |
| NW_003613748v1 | 2170942 |
| NW_003613752v1 | 1191531 |
| NW_003613752v1 | 1216799 |
| NW_003613752v1 | 1400334 |
| NW_003613753v1 | 1568292 |
| NW_003613762v1 | 263819 |
| NW_003613762v1 | 264047 |
| NW_003613762v1 | 632578 |
| NW_003613765v1 | 1696216 |
| NW_003613769v1 | 351010 |
| NW_003613770v1 | 19306 |
| NW_003613772v1 | 119151 |
| NW_003613773v1 | 770426 |
| NW_003613773v1 | 1113201 |
| NW_003613774v1 | 593222 |
| NW_003613774v1 | 1828958 |
| NW_003613777v1 | 506973 |
| NW_003613777v1 | 507220 |
| NW_003613777v1 | 507226 |
| NW_003613778v1 | 1068813 |
| NW_003613780v1 | 425775 |
| NW_003613780v1 | 1653187 |
| NW_003613781v1 | 1141971 |
| NW_003613784v1 | 995335 |
| NW_003613785v1 | 1020545 |
| NW_003613786v1 | 1088987 |
| NW_003613787v1 | 1351063 |
| NW_003613788v1 | 153192 |
| NW_003613790v1 | 857661 |
| NW_003613794v1 | 2093572 |
| NW_003613796v1 | 13723 |
| NW_003613796v1 | 13899 |
| NW_003613796v1 | 451559 |
| NW_003613796v1 | 451596 |
| NW_003613796v1 | 1392151 |
| NW_003613796v1 | 13 [symbol] 264 [symbol] |
| NW_003613797v1 | 823453 |
| NW_003613797v1 | 823472 |
| NW_003613798v1 | 13 [symbol] 7506 |
| NW_003613799v1 | 395268 |
| NW_003613799v1 | 435150 |
| NW_003613799v1 | 1003489 |
| NW_003613799v1 | 1344173 |
| NW_003613799v1 | 1364189 |
| NW_003613799v1 | 1549572 |
| NW_003613799v1 | 1705548 |
| NW_003613799v1 | 1735514 |
| NW_003613801v1 | 963790 |
| NW_003613801v1 | 1192040 |
| NW_003613801v1 | 1309065 |
| NW_003613801v1 | 1379414 |
| NW_003613803v1 | 1077135 |
| NW_003613803v1 | 1195382 |
| NW_003613803v1 | [symbol] 7 [symbol] 472 |
| NW_003613808v1 | 1138048 |
| NW_003613809v1 | 1352281 |
| NW_003613810v1 | 815737 |
| NW_003613815v1 | 972731 |
| NW_003613816v1 | 589224 |
| NW_003613816v1 | 200914 |
| NW_003613820v1 | 508616 |
| NW_003613821v1 | 161961 |
| NW_003613826v1 | 426372 |
| NW_003613830v1 | 9 [symbol] 93 |
| NW_003613830v1 | 6 [symbol] 1306 |
| NW_003613830v1 | 905642 |
| NW_003613830v1 | 1384904 |
| NW_003613830v1 | 1384935 |
| NW_003613830v1 | 1384955 |
| NW_003613830v1 | 1431831 |
| NW_003613831v1 | 1528 [symbol] 7 |
| NW_003613831v1 | 152877 |
| NW_003613833v1 | 235932 |
| NW_003613833v1 | 787668 |
| NW_003613838v1 | 730057 |
| NW_003613838v1 | 763556 |
| NW_003613838v1 | 995488 |
| NW_003613838v1 | 1655742 |
| NW_003613839v1 | 679094 |
| NW_003613842v1 | 4 [symbol] 761 |
| NW_003613842v1 | 687895 |
| NW_003613843v1 | 1003763 |
| NW_003613844v1 | 944148 |
| NW_003613846v1 | 1493678 |
| NW_003613846v1 | 16 [symbol] 17 [symbol] 8 |
| NW_003613846v1 | 174 [symbol] 482 |
| NW_003613846v1 | 1764031 |
| NW_003613847v1 | 1522660 |
| NW_003613849v1 | 1128953 |
| NW_003613852v1 | 1 [symbol] 983 |
| NW_003613852v1 | 186410 |
| NW_003613852v1 | 1399731 |
| NW_003613854v1 | 309513 |
| NW_003613854v1 | 595435 |
| NW_003613854v1 | 946162 |
| NW_003613854v1 | 14467 [symbol] |
| NW_003613855v1 | 1827553 |
| NW_003613856v1 | 759 [symbol] 27 |
| NW_003613856v1 | 1540300 |
| NW_003613857v1 | 822850 |
| NW_003613861v1 | 91038 |
| NW_003613861v1 | 1073457 |
| NW_003613862v1 | 186379 |
| NW_003613862v1 | 216649 |
| NW_003613862v1 | 631543 |
| NW_003613864v1 | 1214869 |
| NW_003613865v1 | 189557 |
| NW_003613865v1 | 395 [symbol] 10 |
| NW_003613865v1 | 1027596 |

[symbol]

indicates data missing or illegible when filed

TABLE 5c
901 CpG sites from CHO cells relevant for the method
according to any aspect of the present invention.

| Chrom | Position |
|---|---|
| NW_003613865v1 | 1304969 |
| NW_003613871v1 | 145191 |
| NW_003613871v1 | 584833 |
| NW_003613871v1 | 914596 |
| NW_003613875v1 | 946546 |
| NW_003613875v1 | 1048831 |
| NW_003613875v1 | 1181684 |
| NW_003613879v1 | 1423339 |
| NW_003613880v1 | 93440 |
| NW_003613884v1 | 378768 |
| NW_003613884v1 | 638322 |
| NW_003613885v1 | 1510598 |
| NW_003613887v1 | 841029 |
| NW_003613890v1 | 818111 |
| NW_003613896v1 | 1658704 |
| NW_003613898v1 | 445164 |
| NW_003613898v1 | 768011 |
| NW_003613899v1 | 13112 |
| NW_003613899v1 | 715664 |
| NW_003613899v1 | 957207 |
| NW_003613899v1 | 957263 |
| NW_003613899v1 | 957352 |
| NW_003613899v1 | 1225021 |
| NW_003613899v1 | 1669864 |
| NW_003613901v1 | 48 [symbol] 941 |
| NW_003613901v1 | 1224107 |
| NW_003613901v1 | 665713 |
| NW_003613902v1 | 665864 |
| NW_003613902v1 | 752145 |
| NW_003613902v1 | 866701 |
| NW_003613902v1 | 867498 |
| NW_003613902v1 | 1055095 |
| NW_003613904v1 | 823348 |
| NW_003613904v1 | 925443 |
| NW_003613904v1 | 1438588 |
| NW_003613906v1 | 211661 |
| NW_003613908v1 | 1064955 |
| NW_003613908v1 | 1118096 |
| NW_003613908v1 | 1118170 |
| NW_003613911v1 | 66547 |
| NW_003613911v1 | 67056 |
| NW_003619913v1 | 195032 |
| NW_003613916v1 | 480030 |
| NW_003613919v1 | 787773 |
| NW_003613919v1 | 1109067 |
| NW_003613919v1 | 1375593 |
| NW_003613919v1 | 1494560 |
| NW_003613921v1 | 354563 |
| NW_003613921v1 | 354587 |
| NW_003613923v1 | 664563 |
| NW_003613923v1 | 1015965 |
| NW_003613923v1 | 1187332 |
| NW_003613923v1 | 1330763 |
| NW_003613923v1 | 1383912 |
| NW_003613926v1 | 135621 |
| NW_003613928v1 | 256592 |
| NW_003613930v1 | 256531 |
| NW_003613933v1 | 650815 |
| NW_003613933v1 | 758871 |
| NW_003613936v1 | 930752 |
| NW_003613936v1 | 1328431 |
| NW_003613941v1 | 366061 |
| NW_003613941v1 | 510987 |
| NW_003613941v1 | 674310 |
| NW_003613941v1 | 808992 |
| NW_003613941v1 | 309022 |
| NW_003613943v1 | 360587 |
| NW_003613943v1 | 1527361 |
| NW_003613943v1 | 1527440 |
| NW_003613944v1 | 1197303 |
| NW_003613949v1 | 1443240 |
| NW_003613951v1 | 122260 |
| NW_003613952v1 | 389211 |
| NW_003613953v1 | 1245377 |
| NW_003613954v1 | 992165 |
| NW_003613957v1 | 1369942 |
| NW_003613958v1 | 608767 |
| NW_003613958v1 | 712377 |
| NW_003613960v1 | 1097495 |
| NW_003613960v1 | 1531274 |
| NW_003613964v1 | 420046 |
| NW_003613966v1 | 845857 |
| NW_003613969v1 | 507908 |
| NW_003613973v1 | 1118664 |
| NW_003613978v1 | 621802 |
| NW_003613978v1 | 1116350 |
| NW_003613978v1 | 1231130 |
| NW_003613981v1 | 731291 |
| NW_003613984v1 | 412098 |
| NW_003613984v1 | 644313 |
| NW_003613985v1 | 19629 |
| NW_003613986v1 | 348489 |
| NW_003613990v1 | 488683 |
| NW_003613993v1 | 446547 |
| NW_003614009v1 | 112436 |
| NW_003614012v1 | 171371 |
| NW_003614012v1 | 171545 |
| NW_003614013v1 | 96889 |
| NW_003614013v1 | 887265 |
| NW_003614013v1 | 1315159 |
| NW_003614015v1 | 98179 |
| NW_003614015v1 | 564529 |
| NW_003614018v1 | 942981 |
| NW_003614028v1 | 891518 |
| NW_003614029v1 | 928930 |
| NW_003614031v1 | 787981 |
| NW_003614033v1 | 332215 |
| NW_003614036v1 | 342657 |
| NW_003614042v1 | 777753 |
| NW_003614042v1 | 1016918 |
| NW_003614042v1 | 1017093 |
| NW_003614043v1 | 312877 |
| NW_003614046v1 | 442298 |
| NW_003614046v1 | 442761 |
| NW_003614046v1 | 564714 |
| NW_003614050v1 | 422741 |
| NW_003614051v1 | 233832 |
| NW_003614053v1 | 176606 |
| NW_003614056v1 | 1033730 |
| NW_003614059v1 | 998277 |
| NW_003614068v1 | 1205689 |
| NW_003614071v1 | 286197 |
| NW_003614071v1 | 286202 |
| NW_003614071v1 | 286263 |
| NW_003614071v1 | 305805 |
| NW_003614071v1 | 686641 |
| NW_003614075v1 | 34077 |
| NW_003614077v1 | 377598 |
| NW_003614077v1 | 378117 |
| NW_003614077v1 | 454224 |
| NW_003614077v1 | 1237946 |
| NW_003614078v1 | 121146 |
| NW_003614078v1 | 378077 |
| NW_003614078v1 | 1102350 |
| NW_003614078v1 | 1209813 |
| NW_003614082v1 | 84020 |
| NW_003614082v1 | 497482 |
| NW_003614085v1 | 398446 |
| NW_003614087v1 | [symbol] 48241 |
| NW_003614095v1 | 256250 |
| NW_003614098v1 | 786600 |
| NW_003614101v1 | 468582 |
| NW_003614105v1 | 326443 |
| NW_003614105v1 | 326477 |
| NW_003614107v1 | 917963 |
| NW_003614108v1 | 152340 |
| NW_003614116v1 | 136201 |
| NW_003614116v1 | 287863 |
| NW_003614116v1 | 846346 |
| NW_003614122v1 | 1148217 |
| NW_003614123v1 | 727828 |
| NW_003614124v1 | 827226 |
| NW_003614126v1 | 124363 |
| NW_003614128v1 | 106493 |
| NW_003614132v1 | 436088 |
| NW_003614137v1 | 36729 |
| NW_003614139v1 | 319672 |
| NW_003614142v1 | 38418 |
| NW_003614145v1 | 60933 |
| NW_003614150v1 | 155767 |
| NW_003614150v1 | 562122 |
| NW_003614150v1 | 6 [symbol] 0803 |
| NW_003614162v1 | 203986 |
| NW_003614162v1 | 203995 |
| NW_003614167v1 | 679649 |
| NW_003614167v1 | 679704 |
| NW_003614172v1 | 174533 |
| NW_003614178v1 | 356967 |
| NW_003614180v1 | 629941 |
| NW_003614183v1 | 361225 |
| NW_003614184v1 | 701032 |
| NW_003614184v1 | 701439 |
| NW_003614187v1 | 666306 |
| NW_003614192v1 | 30918 |
| NW_003614193v1 | 436806 |
| NW_003614195v1 | 640543 |

[symbol]

indicates data missing or illegible when filed

TABLE 5d
901 CpG sites from CHO cells relevant for the method
according to any aspect of the present invention.

| Chrom | Position |
|---|---|
| NW_003614196v1 | 194871 |
| NW_003614196v1 | 194945 |
| NW_003614196v1 | 227339 |
| NW_003614196v1 | 227396 |
| NW_003614196v1 | 227438 |
| NW_003614199v1 | 803543 |
| NW_003614206v1 | 71083 |
| NW_003614208v1 | 838916 |
| NW_003614213v1 | 253851 |
| NW_003614215v1 | 120196 |
| NW_003614215v1 | 110224 |
| NW_003614216v1 | 860684 |
| NW_003614217v1 | 274712 |
| NW_003614217v1 | 370513 |
| NW_003614217v1 | 664205 |
| NW_003614217v1 | 786400 |
| NW_003614218v1 | 790833 |
| NW_003614222v1 | 663535 |
| NW_003614223v1 | 153022 |
| NW_003614224v1 | 748450 |
| NW_003614228v1 | 870930 |
| NW_003614229v1 | 658299 |
| NW_003614234v1 | 16832 |
| NW_003614243v1 | 655322 |
| NW_003614244v1 | 919362 |
| NW_003614244v1 | 927999 |
| NW_003614244v1 | 928016 |
| NW_003614244v1 | 9280 [symbol] 4 |
| NW_003614247v1 | 631877 |
| NW_003614255v1 | 512391 |
| NW_003614257v1 | 438966 |
| NW_003614258v1 | 209828 |
| NW_003614268v1 | 787478 |
| NW_003614269v1 | 226296 |
| NW_003614273v1 | 101841 |
| NW_003614274v1 | 116488 |
| NW_003614274v1 | 832150 |
| NW_003614276v1 | 106154 |
| NW_003614300v1 | 610517 |
| NW_003614301v1 | 254236 |
| NW_003614301v1 | 347491 |
| NW_003614302v1 | 411089 |
| NW_003614302v1 | 701423 |
| NW_003614320v1 | 215637 |
| NW_003614321v1 | [symbol] 46617 |
| NW_003614322v1 | 134058 |
| NW_003614327v1 | 502461 |
| NW_003614327v1 | 502854 |
| NW_003614327v1 | 502856 |
| NW_003614330v1 | 629913 |
| NW_003614332v1 | 730966 |
| NW_003614337v1 | 220020 |
| NW_003614338v1 | 154838 |
| NW_003614338v1 | 194501 |
| NW_003614338v1 | 194567 |
| NW_003614338v1 | 212084 |
| NW_003614338v1 | 212456 |
| NW_003614338v1 | 541042 |
| NW_003614339v1 | 19296 |
| NW_003614339v1 | 373989 |
| NW_003614339v1 | 603502 |
| NW_003614339v1 | 604126 |
| NW_003614340v1 | 372195 |
| NW_003614349v1 | 667838 |
| NW_003614353v1 | 603156 |
| NW_003614356v1 | 585773 |
| NW_003614359v1 | 349056 |
| NW_003614359v1 | 662100 |
| NW_003614362v1 | 143969 |
| NW_003614383v1 | 27499 |
| NW_003614383v1 | 646586 |
| NW_003614393v1 | 451814 |
| NW_003614393v1 | 468734 |
| NW_003614393v1 | 585923 |
| NW_003614393v1 | 585954 |
| NW_003614393v1 | 677405 |
| NW_003614394v1 | 102487 |
| NW_003614397v1 | 70007 |
| NW_003614409v1 | 369487 |
| NW_003614410v1 | 12092 |
| NW_003614410v1 | 622347 |
| NW_003614411v1 | 176563 |
| NW_003614411v1 | 190968 |
| NW_003614411v1 | 434169 |
| NW_003614411v1 | 487 [symbol] 0 |
| NW_003614412v1 | 132296 |
| NW_003614428v1 | 187819 |
| NW_003614439v1 | 674939 |
| NW_003614446v1 | 700099 |
| NW_003614461v1 | 55877 |
| NW_003614462v1 | 97543 |
| NW_003614462v1 | 700703 |
| NW_003614478v1 | 31537 |
| NW_003614478v1 | 265185 |
| NW_003614479v1 | 236957 |
| NW_003614479v1 | 704516 |
| NW_003614483v1 | 162449 |
| NW_003614488v1 | 605141 |
| NW_003614491v1 | 108302 |
| NW_003614499v1 | 400281 |
| NW_003614504v1 | 440486 |
| NW_003614510v1 | 544086 |
| NW_003614512v1 | 135768 |
| NW_003614516v1 | 17908 |
| NW_003614516v1 | 247922 |
| NW_003614517v1 | 100421 |
| NW_003614517v1 | 611252 |
| NW_003614528v1 | 360463 |
| NW_003614544v1 | 442171 |
| NW_003614544v1 | 442199 |
| NW_003614548v1 | 96409 |
| NW_003614548v1 | 584698 |
| NW_003614552v1 | 509163 |
| NW_003614555v1 | 452967 |
| NW_003614555v1 | 453842 |
| NW_003614566v1 | 276561 |
| NW_003614566v1 | 635291 |
| NW_003614566v1 | 649053 |
| NW_003614570v1 | 135512 |
| NW_003614570v1 | 278935 |
| NW_003614570v1 | 309823 |
| NW_003614572v1 | 446459 |
| NW_003614577v1 | 233921 |
| NW_003614577v1 | 233956 |
| NW_003614577v1 | 233963 |
| NW_003614589v1 | 53966 |
| NW_003614589v1 | 605911 |
| NW_003614593v1 | 414670 |
| NW_003614594v1 | 35961 |
| NW_003614594v1 | 35966 |
| NW_003614607v1 | 403228 |
| NW_003614612v1 | 82988 |
| NW_003614613v1 | 356004 |
| NW_003614660v1 | 428031 |
| NW_003614665v1 | 268428 |
| NW_003614665v1 | 268437 |
| NW_003614665v1 | 493779 |
| NW_003614668v1 | 306003 |
| NW_003614679v1 | 60979 |
| NW_003614681v1 | 127703 |
| NW_003614681v1 | 531347 |
| NW_003614681v1 | 531372 |
| NW_003614682v1 | 290991 |
| NW_003614682v1 | 356406 |
| NW_003614690v1 | 174989 |
| NW_003614712v1 | 448204 |
| NW_003614714v1 | 500703 |
| NW_003614720v1 | 165755 |
| NW_003614722v1 | 480821 |
| NW_003614726v1 | 370691 |
| NW_003614736v1 | 523511 |
| NW_003614744v1 | 357937 |
| NW_003614744v1 | 357959 |
| NW_003614747v1 | 449768 |
| NW_003614760v1 | 309418 |
| NW_003614776v1 | 68048 |
| NW_003614791v1 | 192769 |
| NW_003614794v1 | 167397 |
| NW_003614796v1 | 381920 |
| NW_003614797v1 | 256799 |
| NW_003614797v1 | 360535 |
| NW_003614798v1 | 204988 |
| NW_003614798v1 | 369430 |
| NW_003614801v1 | 423551 |
| NW_003614801v1 | 423574 |
| NW_003614819v1 | 282077 |
| NW_003614840v1 | 72528 |
| NW_003614845v1 | 146523 |
| NW_003614852v1 | 404813 |
| NW_003614853v1 | 391040 |
| NW_003614860v1 | 243046 |
| NW_003614860v1 | 424380 |
| NW_003614866v1 | 361892 |
| NW_003614867v1 | 406779 |
| NW_003614868v1 | 156934 |
| NW_003614870v1 | 400 |

[symbol]

indicates data missing or illegible when filed

TABLE 5e
901 CpG sites from CHO cells relevant for the method
according to any aspect of the present invention.

| Chrom | Position |
|---|---|
| NW_003614870v1 | 262527 |
| NW_003614875v1 | 184242 |
| NW_003614875v1 | 243737 |
| NW_003614892v1 | 160821 |
| NW_003614895v1 | 323224 |
| NW_003614897v1 | 187043 |
| NW_003614897v1 | 254531 |
| NW_003614899v1 | 258250 |
| NW_003614903v1 | 306933 |
| NW_003614905v1 | 58205 |
| NW_003614917v1 | 193346 |
| NW_003614928v1 | 199144 |
| NW_003614933v1 | 177106 |
| NW_003614943v1 | 199676 |
| NW_003614943v1 | 242401 |
| NW_003614949v1 | 207266 |
| NW_003614949v1 | 377748 |
| NW_003614955v1 | 166432 |
| NW_003614969v1 | 322133 |
| NW_003614971v1 | 320376 |
| NW_003614984v1 | 91798 |
| NW_003614997v1 | 35543 |
| NW_003615000v1 | 27910 |
| NW_003615000v1 | 28068 |
| NW_003615003v1 | 269591 |
| NW_003615006v1 | 5994 |
| NW_003615007v1 | 111922 |
| NW_003615014v1 | 154435 |
| NW_003615015v1 | 6155 |
| NW_003615015v1 | 260913 |
| NW_003615023v1 | 2228 |
| NW_003615030v1 | 27086 |
| NW_003615035v1 | 237300 |
| NW_003615041v1 | 357621 |
| NW_003615050v1 | 322583 |
| NW_003615059v1 | 40144 |
| NW_003615059v1 | 233070 |
| NW_003615059v1 | 360743 |
| NW_003615063v1 | 368059 |
| NW_003615068v1 | 295953 |
| NW_003615068v1 | 295988 |
| NW_003615071v1 | 88416 |
| NW_003615087v1 | 100330 |
| NW_003615094v1 | 329749 |
| NW_003615109v1 | 180996 |
| NW_003615112v1 | 314834 |
| NW_003615112v1 | 323169 |
| NW_003615132v1 | 211606 |
| NW_003615134v1 | 298559 |
| NW_003615134v1 | 315993 |
| NW_003615137v1 | 45483 |
| NW_003615140v1 | 57619 |
| NW_003615153v1 | 288865 |
| NW_003615154v1 | 185621 |
| NW_003615165v1 | 272212 |
| NW_003615169v1 | 126930 |
| NW_003615178v1 | 286304 |
| NW_003615185v1 | 286742 |
| NW_003615189v1 | 63546 |
| NW_003615199v1 | 280773 |
| NW_003615211v1 | 210266 |
| NW_003615220v1 | 138675 |
| NW_003615225v1 | 145794 |
| NW_003615246v1 | 248694 |
| NW_003615247v1 | 112422 |
| NW_003615257v1 | 51634 |
| NW_003615296v1 | 4500 |
| NW_003615310v1 | 172562 |
| NW_003615317v1 | 157202 |
| NW_003615327v1 | 226408 |
| NW_003615346v1 | 36906 |
| NW_003615352v1 | 142552 |
| NW_003615387v1 | 237201 |
| NW_003615402v1 | 160872 |
| NW_003615402v1 | 160901 |
| NW_003615404v1 | 237437 |
| NW_003615408v1 | 160049 |
| NW_003615411v1 | 50696 |
| NW_003615425v1 | 137861 |
| NW_003615425v1 | 137869 |
| NW_003615432v1 | 156296 |
| NW_003615438v1 | 91537 |
| NW_003615442v1 | 61527 |
| NW_003615454v1 | 11729 |
| NW_003615466v1 | 127062 |
| NW_003615469v1 | 181462 |
| NW_003615469v1 | 212431 |
| NW_003615496v1 | 11623 |
| NW_003615506v1 | 18763 |
| NW_003615506v1 | 41455 |
| NW_003615517v1 | 54560 |
| NW_003615564v1 | 130901 |
| NW_003615635v1 | 62135 |
| NW_003615648v1 | 70641 |
| NW_003615656v1 | 98072 |
| NW_003615668v1 | 9998 |
| NW_003615668v1 | 191604 |
| NW_003615732v1 | 125239 |
| NW_003615739v1 | 167355 |
| NW_003615768v1 | 47566 |
| NW_003615769v1 | 174203 |
| NW_003615772v1 | 80763 |
| NW_003615791v1 | 28529 |
| NW_003615850v1 | 71253 |
| NW_003615864v1 | 84657 |
| NW_003615871v1 | 134996 |
| NW_003615896v1 | 110801 |
| NW_003615968v1 | 63569 |
| NW_003615987v1 | 8929 |
| NW_003615992v1 | 47499 |
| NW_003615992v1 | 52086 |
| NW_003616010v1 | 136310 |
| NW_003616064v1 | 42367 |
| NW_003616071v1 | 132545 |
| NW_003616073v1 | 63880 |
| NW_003616073v1 | 91830 |
| NW_003616083v1 | 94525 |
| NW_003616107v1 | 106696 |
| NW_003616184v1 | 49897 |
| NW_003616188v1 | 87387 |
| NW_003616190v1 | 59633 |
| NW_003616203v1 | 143560 |
| NW_003616203v1 | 143855 |
| NW_003616210v1 | 29298 |
| NW_003616218v1 | 5002 |
| NW_003616251v1 | 28599 |
| NW_003616251v1 | 102846 |
| NW_003616251v1 | 102869 |
| NW_003616270v1 | 72174 |
| NW_003616289v1 | 34015 |
| NW_003616314v1 | 12286 |
| NW_003616314v1 | 120792 |
| NW_003616392v1 | 23313 |
| NW_003616392v1 | 78238 |
| NW_003616417v1 | 57231 |
| NW_003616422v1 | 15008 |
| NW_003616425v1 | 21105 |
| NW_003616443v1 | 100341 |
| NW_003616480v1 | 56275 |
| NW_003616489v1 | 73161 |
| NW_003616508v1 | 38678 |
| NW_003616594v1 | 73420 |
| NW_003616626v1 | 631 |
| NW_003616640v1 | 70568 |
| NW_003616688v1 | 103504 |
| NW_003616693v1 | 51251 |
| NW_003616698v1 | 79159 |
| NW_003616758v1 | 90993 |
| NW_003616801v1 | 63849 |
| NW_003616838v1 | 8964 |
| NW_003616838v1 | 41693 |
| NW_003616892v1 | 42331 |
| NW_003616892v1 | 42442 |
| NW_003616939v1 | 86664 |
| NW_003616941v1 | 20316 |
| NW_003616941v1 | 20347 |
| NW_003616990v1 | 3012 |
| NW_003616995v1 | 87853 |
| NW_003617063v1 | 37471 |
| NW_003617063v1 | 42308 |
| NW_003617069v1 | 43235 |
| NW_003617109v1 | 6216 |
| NW_003617129v1 | 58146 |
| NW_003617137v1 | 13841 |
| NW_003617180v1 | 29054 |
| NW_003617202v1 | 54308 |
| NW_003617226v1 | 51068 |
| NW_003617243v1 | 41546 |
| NW_003617289v1 | 32716 |
| NW_003617297v1 | 12962 |
| NW_003617301v1 | 10074 |
| NW_003617336v1 | 6770 |
| NW_003617389v1 | 38213 |
| NW_003617444v1 | 23253 |
| NW_003617466v1 | 35192 |
| NW_003617863v1 | 46437 |

TABLE 5f
901 CpG sites from CHO cells relevant for the method
according to any aspect of the present invention.

| Chrome | Position |
|---|---|
| NW_003617894v1 | 20022 |
| NW_003617963v1 | 33815 |
| NW_003618119v1 | 38094 |
| NW_003618301v1 | 9512 |
| NW_003618434v1 | 23262 |
| NW_003618516v1 | 18003 |
| NW_003620998v1 | 2344 |
| NW_003623627v1 | 2564 |
| NW_003624766v1 | 2494 |
| NW_003625307v1 | 566 |
| NW_003625521v1 | 991 |
| NW_003625629v1 | 669 |
| NW_003627899v1 | 1014 |
| NW_003629119v1 | 843 |
| NW_003629198v1 | 864 |
| NW_003630387v1 | 696 |
| NW_003630387v1 | 986 |
| NW_003630387v1 | 1010 |
| NW_003656587v1 | 203 |
| NW_003613635v1 | 608730 |

1. A method of determining suitability of at least one Chinese Hamster Ovary (CHO) test cell line for optimal heterologous protein production, the method comprising:
(a) determining a test methylation profile from genomic material obtained from the CHO test cell line; and
(b) comparing the test methylation profile obtained from (a) with a reference methylation profile, wherein the reference methylation profile comprises the methylation status of more than one CpG site from at least one CHO reference cell line that displays at least one phenotype of interest for optimal heterologous protein production,
wherein a significant similarity in the test methylation profile of (a) compared to the reference methylation profile, is indicative of the CHO test cell line being suitable for optimal heterologous protein production,
wherein the test methylation profile and reference methylation profile are from CpG sites from the CHO cell genome and are determined using DNA methylation-bead-based array.

2. The method according to claim 1, wherein the reference methylation profile is a compilation of more than one CpG site from at least one CHO reference cell line that displays at least one phenotype of interest for optimal heterologous protein production.

3. The method according to claim 1, wherein the phenotype of interest for optimal heterologous protein is selected from the group consisting of phenotypic homogeneity, protein productivity, and protein quality.

4. A method of selecting at least one CHO cell comprising a phenotype of interest from a population of CHO cells from a parental clone, the method comprising the steps of:
(a) determining a test methylation profile from genomic material obtained from the CHO cell, and
(b) comparing the test methylation profile of (a) with a reference methylation profile from a parental clone displaying the phenotype of interest,
wherein a significant similarity between the test methylation profile and the reference methylation profile of (b) is indicative of the cell having the phenotype of interest of the parental clone;
wherein the test methylation profile and reference methylation profile are from CpG sites from the CHO cell genome and are determined using DNA methylation-bead-based array; and
wherein the phenotype of interest is selected from the group consisting of phenotypic homogeneity, protein productivity, and protein quality.

5. A method of identifying at least one CHO test cell line that is capable of producing at least one biosimilar relative to a heterologous protein produced by a CHO reference cell line, the method comprising the steps of:
(a) determining a test methylation profile from genomic material obtained from the CHO test cell line, and
(b) comparing the test methylation profile of (a) with the reference methylation profile of the CHO reference cell line,
wherein a significant similarity between the test methylation profile of (a) and the reference methylation profile is indicative of the two cell lines producing biosimilars; and
wherein the test methylation profile and reference methylation profile are from CpG sites from the CHO cell genome and are determined using DNA methylation-bead-based array.

6. A method of identifying at least one CHO test cell line that is capable of producing at least one bio-identical relative to a heterologous protein produced by a CHO reference cell line, the method comprising the steps of:
(a) determining a test methylation profile from genomic material obtained from the CHO test cell line, and
(b) comparing the test methylation profile of (a) with the reference methylation profile of the CHO reference cell line,
wherein when the test methylation profile of (a) and the reference methylation profile are identical, it is indicative of the two cell lines producing bio-identicals; and
wherein the test methylation profile and reference methylation profile are from CpG sites from the CHO cell genome and are determined using DNA methylation-bead-based array.

7. A method for assessing one or more phenotypic parameters of at least one test CHO cell line, the method comprising the steps of
(a) determining a test methylation status of one or more pre-selected methylation sites from the genomic material obtained from the test CHO cell line;
(b) determining from the methylation status determined in (a) a test methylation profile of the test CHO cell line; and
(c) comparing the test methylation profile determined in (b) with at least one predetermined reference methylation profiles, wherein each of the predetermined reference methylation profiles is specific for a reference CHO cell line with at least one phenotypic parameter;
wherein if the test methylation profile is significantly similar to one of the predetermined reference methylation profiles, the test CHO cell line has similar, or preferably the same phenotypic parameter as the reference CHO cell line with the predetermined reference methylation profile; and
wherein the test methylation profile and reference methylation profile are from CpG sites from the CHO cell genome and are determined using DNA methylation-bead-based array.

8. The method according to claim 7, wherein the phenotypic parameter is selected from the group consisting of: optimal carbohydrate metabolism, optimal amino acid metabolism, optimal lipid metabolism, optimal protein productivity; and optimal cell survivability.

9. A method for developing a test system for determining if a test CHO cell line is capable of optimal heterologous protein production, the method comprising the steps of:
(a) determining a test methylation status of one or more pre-selected methylation sites from the genomic material obtained from the test CHO cell line;
(b) selecting from the pre-selected methylation sites a reference panel of methylation sites which is characterized by a specific and distinct differential methylation profile for each phenotypic parameter or phenotype of interest;
(c) obtaining a test system by assigning a reference methylation profile for each of the phenotypic parameter or phenotypes of interest; and
wherein a comparison of a test methylation profile obtained from a test sample with the reference methylation profiles obtained in (c) allows for confirming if the test CHO cell line is capable of optimal heterologous protein production; and
wherein the test methylation profile and reference methylation profile are from CpG sites from the CHO cell genome and are determined using DNA methylation-bead-based array.

10. A method of determining if a CHO cell line is robust, stable and capable of optimal heterologous protein production before introduction of a transgene into the cell, the method comprising the steps of:
(a) determining a methylation profile from genomic material obtained from the CHO cell line; and
(b) comparing the methylation profile of (a), with a reference methylation profile for a CHO cell line that is robust, stable and capable of optimal heterologous protein production,
wherein a significant similarity between the test methylation profile of (a) and the reference methylation profile is indicative of the CHO cell line being robust, stable and capable of optimal heterologous protein production; and
wherein the test methylation profile and reference methylation profile are from CpG sites from the CHO cell genome and are determined using DNA methylation-bead-based array.

11. The method according to claim 1, wherein the CpG sites comprise at least one of the CpG sites provided in Tables 5a-5f.

12. A method of determining regulation of transgene expression in at least one CHO cell line genetically modified with the transgene, the method comprising the step of:
measuring the methylation level of at least one CpG site of at least one viral promoter of the transgene, and
wherein the DNA methylation level is determined using a bead-based DNA methylation-array.

13. A DNA bead based methylation array comprising at least:
a plurality of distinct locations, each location having at least one probe molecule comprising a nucleic acid sequence complementary to a plurality of CpG sites of a CHO cell,
wherein the CpG sites of the CHO cell are at least selected from the Tables 5a-5f.
